SEQUENCE LISTING (20 sequences)

SEQ ID NO: 1 - protein, 58 aa, synthetic construct
IAAQFDKIAQIAFYAILHLPNLTEEQRHAFIQSLRDDPSVSKEILAEAKKLNDAQAPK

SEQ ID NO: 2 - protein, 58 aa, synthetic construct
IAAQFDKIQQAAFYAILHLPNLTEEQRHAFIQSLRDDPSVSKEILAEAKKLNDAQAPK

SEQ ID NO: 3 - protein, 58 aa, synthetic construct
IAAQFDKIAQAAFYEILHLPNLTEEQRHAFIQSLRDDPSVSKEILAEAKKLNDAQAPK

SEQ ID NO: 4 - protein, 58 aa, synthetic construct
IAAQFDKIQQIAFYEILHLPNLTEEQRHAFIQSLRDDPSVSKEILAEAKKLNDAQAPK

SEQ ID NO: 5 - protein, 58 aa, synthetic construct
IAAQFDKIAQAAFYAILHLPNLTEEQRHAFIQSLRDDPSVSKEILAEAKKLNDAQAPK

SEQ ID NO: 6 - protein, 58 aa, synthetic construct
IAAQFDKIQQAAFYEILHLPNLTEEQRHAFIQSLRDDPSVSKEILAEAKKLNDAQAPK

SEQ ID NO: 7 - protein, 288 aa, synthetic construct
AQFDKIAQIAFYAILHLPNLTEEQRHAFIQSLRDDPSVSKEILAEAKKLNDAQAPKIAAQFDKIAQIAFYAILHLPNLTEEQRHAFIQSLRDDPSVSKEILAEAKKLNDAQAPKIAAQFDKIAQIAFYAILHLPNLTEEQRHAFIQSLRDDPSVSKEILAEAKKLNDAQAPKIAAQFDKIAQIAFYAILHLPNLTEEQRHAFIQSLRDDPSVSKEILAEAKKLNDAQAPKIAAQFDKIAQIAFYAILHLPNLTEEQRHAFIQSLRDDPSVSKEILAEAKKLNDAQAPK

SEQ ID NO: 8 - protein, 288 aa, synthetic construct
AQFDKIQQAAFYAILHLPNLTEEQRHAFIQSLRDDPSVSKEILAEAKKLNDAQAPKIAAQFDKIQQAAFYAILHLPNLTEEQRHAFIQSLRDDPSVSKEILAEAKKLNDAQAPKIAAQFDKIQQAAFYAILHLPNLTEEQRHAFIQSLRDDPSVSKEILAEAKKLNDAQAPKIAAQFDKIQQAAFYAILHLPNLTEEQRHAFIQSLRDDPSVSKEILAEAKKLNDAQAPKIAAQFDKIQQAAFYAILHLPNLTEEQRHAFIQSLRDDPSVSKEILAEAKKLNDAQAPK

SEQ ID NO: 9 - protein, 288 aa, synthetic construct
AQFDKIAQAAFYEILHLPNLTEEQRHAFIQSLRDDPSVSKEILAEAKKLNDAQAPKIAAQFDKIAQAAFYEILHLPNLTEEQRHAFIQSLRDDPSVSKEILAEAKKLNDAQAPKIAAQFDKIAQAAFYEILHLPNLTEEQRHAFIQSLRDDPSVSKEILAEAKKLNDAQAPKIAAQFDKIAQAAFYEILHLPNLTEEQRHAFIQSLRDDPSVSKEILAEAKKLNDAQAPKIAAQFDKIAQAAFYEILHLPNLTEEQRHAFIQSLRDDPSVSKEILAEAKKLNDAQAPK

SEQ ID NO: 10 - protein, 288 aa, synthetic construct
AQFDKIQQIAFYEILHLPNLTEEQRHAFIQSLRDDPSVSKEILAEAKKLNDAQAPKIAAQFDKIQQIAFYEILHLPNLTEEQRHAFIQSLRDDPSVSKEILAEAKKLNDAQAPKIAAQFDKIQQIAFYEILHLPNLTEEQRHAFIQSLRDDPSVSKEILAEAKKLNDAQAPKIAAQFDKIQQIAFYEILHLPNLTEEQRHAFIQSLRDDPSVSKEILAEAKKLNDAQAPKIAAQFDKIQQIAFYEILHLPNLTEEQRHAFIQSLRDDPSVSKEILAEAKKLNDAQAPK

SEQ ID NO: 11 - protein, 288 aa, synthetic construct
AQFDKIAQAAFYAILHLPNLTEEQRHAFIQSLRDDPSVSKEILAEAKKLNDAQAPKIAAQFDKIAQAAFYAILHLPNLTEEQRHAFIQSLRDDPSVSKEILAEAKKLNDAQAPKIAAQFDKIAQAAFYAILHLPNLTEEQRHAFIQSLRDDPSVSKEILAEAKKLNDAQAPKIAAQFDKIAQAAFYAILHLPNLTEEQRHAFIQSLRDDPSVSKEILAEAKKLNDAQAPKIAAQFDKIAQAAFYAILHLPNLTEEQRHAFIQSLRDDPSVSKEILAEAKKLNDAQAPK

SEQ ID NO: 12 - protein, 288 aa, synthetic construct
AQFDKIQQAAFYEILHLPNLTEEQRHAFIQSLRDDPSVSKEILAEAKKLNDAQAPKIAAQFDKIQQAAFYEILHLPNLTEEQRHAFIQSLRDDPSVSKEILAEAKKLNDAQAPKIAAQFDKIQQAAFYEILHLPNLTEEQRHAFIQSLRDDPSVSKEILAEAKKLNDAQAPKIAAQFDKIQQAAFYEILHLPNLTEEQRHAFIQSLRDDPSVSKEILAEAKKLNDAQAPKIAAQFDKIQQAAFYEILHLPNLTEEQRHAFIQSLRDDPSVSKEILAEAKKLNDAQAPK

SEQ ID NO: 13 - protein, 58 aa, synthetic construct
  VARIANT 9: note = X can be any amino acid, preferably X is Q or A
  VARIANT 11: note = X can be any amino acid, preferably X is A or I
  VARIANT 15: note = X can be any amino acid, preferably X is A orE
IAAQFDKIXQXAFYXILHLPNLTEEQRHAFIQSLRDDPSVSKEILAEAKKLNDAQAPK

SEQ ID NO: 14 - protein, 288 aa, synthetic construct
  VARIANT 7: note = X can be any amino acid, preferably X is Q or A
  VARIANT 9: note = X can be any amino acid, preferably X is A or I
  VARIANT 13: note = X can be any amino acid, preferably X is A or E
  VARIANT 65: note = X can be any amino acid, preferably X is Q or A
  VARIANT 67: note = X can be any amino acid, preferably X is A or I
  VARIANT 71: note = X can be any amino acid, preferably X is A or E
  VARIANT 123: note = X can be any amino acid, preferably X is Q or A
  VARIANT 125: note = X can be any amino acid, preferably X is A or I
  VARIANT 129: note = X can be any amino acid, preferably X is A or E
  VARIANT 181: note = X can be any amino acid, preferably X is Q or A
  VARIANT 183: note = X can be any amino acid, preferably X is A or I
  VARIANT 187: note = X can be any amino acid, preferably X is A or E
  VARIANT 239: note = X can be any amino acid, preferably X is Q or A
  VARIANT 241: note = X can be any amino acid, preferably X is A or I
  VARIANT 245: note = X can be any amino acid, preferably X is A or E
AQFDKIXQXAFYXILHLPNLTEEQRHAFIQSLRDDPSVSKEILAEAKKLNDAQAPKIAAQFDKIXQXAFYXILHLPNLTEEQRHAFIQSLRDDPSVSKEILAEAKKLNDAQAPKIAAQFDKIXQXAFYXILHLPNLTEEQRHAFIQSLRDDPSVSKEILAEAKKLNDAQAPKIAAQFDKIXQXAFYXILHLPNLTEEQRHAFIQSLRDDPSVSKEILAEAKKLNDAQAPKIAAQFDKIXQXAFYXILHLPNLTEEQRHAFIQSLRDDPSVSKEILAEAKKLNDAQAPK

SEQ ID NO: 15 - protein, 58 aa, synthetic construct
  VARIANT 4: note = X at position 4 may be Q or K
  VARIANT 7: note = X at position 7 may be K or E
  VARIANT 9: note = X at position 9 may be A or Q
  VARIANT 11: note = X at position 11 may be A or I
  VARIANT 15: note = X at position 15 may be A or E
IAAXFDXIXQXAFYXILHLPNLTEEQRHAFIQSLRDDPSVSKEILAEAKKLNDAQAPK

SEQ ID NO: 16 - protein, 288 aa, synthetic construct
  VARIANT 2: note = X at position 2 may be Q or K
  VARIANT 60: note = X at position 60 may be Q or K
  VARIANT 118: note = X at position 118 may be Q or K
  VARIANT 176: note = X at position 176 may be Q or K
  VARIANT 234: note = X at position 234 may be Q or K
  VARIANT 5: note = X at position 5 may be K or E
  VARIANT 63: note = X at position 63 may be K or E
  VARIANT 121: note = X at position 121 may be K or E
  VARIANT 179: note = X at position 179 may be K or E
  VARIANT 237: note = X at position 237 may be K or E
  VARIANT 7: note = X at position 7 may be A or Q
  VARIANT 65: note = X at position 65 may be A or Q
  VARIANT 123: note = X at position 123 may be A or Q
  VARIANT 181: note = X at position 181 may be A or Q
  VARIANT 239: note = X at position 239 may be A or Q
  VARIANT 9: note = X at position 9 may be A or I
  VARIANT 67: note = X at position 67 may be A or I
  VARIANT 125: note = X at position 125 may be A or I
  VARIANT 183: note = X at position 183 may be A or I
  VARIANT 241: note = X at position 241 may be A or I
  VARIANT 13: note = X at position 13 may be A or E
  VARIANT 71: note = X at position 71 may be A or E
  VARIANT 129: note = X at position 129 may be A or E
  VARIANT 187: note = X at position 187 may be A or E
  VARIANT 245: note = X at position 245 may be A or E
AXFDXIXQXAFYXILHLPNLTEEQRHAFIQSLRDDPSVSKEILAEAKKLNDAQAPKIAAXFDXIXQXAFYXILHLPNLTEEQRHAFIQSLRDDPSVSKEILAEAKKLNDAQAPKIAAXFDXIXQXAFYXILHLPNLTEEQRHAFIQSLRDDPSVSKEILAEAKKLNDAQAPKIAAXFDXIXQXAFYXILHLPNLTEEQRHAFIQSLRDDPSVSKEILAEAKKLNDAQAPKIAAXFDXIXQXAFYXILHLPNLTEEQRHAFIQSLRDDPSVSKEILAEAKKLNDAQAPK

SEQ ID NO: 17 - protein, 58 aa, synthetic construct
IAAKFDEIAQAAFYEILHLPNLTEEQRHAFIQSLRDDPSVSKEILAEAKKLNDAQAPK

SEQ ID NO: 18 - protein, 288 aa, synthetic construct
AKFDEIAQAAFYEILHLPNLTEEQRHAFIQSLRDDPSVSKEILAEAKKLNDAQAPKIAAKFDEIAQAAFYEILHLPNLTEEQRHAFIQSLRDDPSVSKEILAEAKKLNDAQAPKIAAKFDEIAQAAFYEILHLPNLTEEQRHAFIQSLRDDPSVSKEILAEAKKLNDAQAPKIAAKFDEIAQAAFYEILHLPNLTEEQRHAFIQSLRDDPSVSKEILAEAKKLNDAQAPKIAAKFDEIAQAAFYEILHLPNLTEEQRHAFIQSLRDDPSVSKEILAEAKKLNDAQAPK

SEQ ID NO: 19 - protein, 58 aa, synthetic construct
IAAQFDKIAQAAFYEILHLPNLTEEQRHAFIQSLRDDPSVSKEILAEAKKLNDAQAPP

SEQ ID NO: 20 - protein, 288 aa, synthetic construct
AQFDKIAQAAFYEILHLPNLTEEQRHAFIQSLRDDPSVSKEILAEAKKLNDAQAPPIAAQFDKIAQAAFYEILHLPNLTEEQRHAFIQSLRDDPSVSKEILAEAKKLNDAQAPPIAAQFDKIAQAAFYEILHLPNLTEEQRHAFIQSLRDDPSVSKEILAEAKKLNDAQAPPIAAQFDKIAQAAFYEILHLPNLTEEQRHAFIQSLRDDPSVSKEILAEAKKLNDAQAPPIAAQFDKIAQAAFYEILHLPNLTEEQRHAFIQSLRDDPSVSKEILAEAKKLNDAQAPK

(19) United States
(12) Patent Application Publication
(10) Pub. No.: US 2026/0209265 A1
(43) Pub. Date: Jul. 23, 2026

(54) NEW LIGANDS FOR AFFINITY CHROMATOGRAPHY

(71) Applicant: Navigo Proteins GmbH, Halle/Saale (DE)

(72) Inventors: Erik Fiedler, Halle/Saale (DE); Ulrich Haupts, Halle/Saale (DE); Heike Boecker, Halle/Saale (DE); Michael Niemeyer, Halle/Saale (DE); Hanna Bobolowski, Halle/Saale (DE)

(21) Appl. No.: 19/139,562

(22) PCT Filed: Dec. 15, 2023
(86) PCT No.: PCT/EP2023/086181

§ 371 Date: Jun. 16, 2025

(30) Foreign Application Priority Data
Dec. 16, 2022 (EP) 22214280.4
Jul. 7, 2023 (EP) 23184276.6

(51) Int. Cl. C07K 1/22; C07K 14/245

(52) U.S. Cl. CPC C07K 1/22; C07K 14/245

(57) ABSTRACT

The present invention relates to novel ligands for affinity chromatography, in particular purification of antibodies. The novel ligands are immunoglobulin (Ig) binding proteins with superior properties for highly efficient purification methods for antibodies (immunoglobulins). The invention further relates to affinity matrices comprising the ligands of the invention. The invention also relates to a use of these Ig binding proteins or affinity matrices for affinity purification of immunoglobulins and to methods of affinity purification using the Ig binding proteins of the invention.

### FIELD OF THE INVENTION

The present invention relates to novel ligands for affinity chromatography, in particular purification of antibodies. The novel ligands are immunoglobulin (Ig) binding proteins with superior properties for highly efficient purification methods for antibodies (immunoglobulins).

The invention further relates to affinity matrices comprising the ligands of the invention. The invention also relates to a use of these Ig binding proteins or affinity matrices for affinity purification of immunoglobulins and to methods of affinity purification using the Ig binding proteins of the invention.

### BACKGROUND OF THE INVENTION

Many biotechnological and pharmaceutical applications require the removal of contaminants from a sample containing antibodies. An established procedure for capturing and purifying antibodies and molecules that contain an Fc-domain is affinity chromatography using the bacterial cell surface Protein A from Staphylococcus aureus as selective ligand for immunoglobulins (see, for example, review by Huse et al., J. Biochem. Biophys. Methods 51, 2002: 217-231). Wild-type Protein A binds to the Fc region of IgG molecules with high affinity and selectivity. Variants of Protein A with improved properties such as alkaline stability are available for purifying antibodies and various chromatographic matrices comprising Protein A ligands are commercially available. However, currently available Protein A based chromatography matrices show a loss of binding capacity for immunoglobulins following exposure to alkaline conditions and require elution conditions at pH below 4.

### Technical Problems Underlying the Invention

Most large scale production processes for antibodies or Fc-containing (fusion) proteins use Protein A for affinity purification. However, due to limitations of Protein A applications in affinity chromatography there is a need in the art to provide novel Ig binding proteins with improved properties that specifically bind to immunoglobulins in order to facilitate affinity purification of immunoglobulins. To maximally exploit the value of the chromatographic matrices comprising Ig binding proteins it is desirable to use the affinity ligand matrices multiple times. Between chromatography cycles a thorough cleaning procedure is required for sanitization and removal of residual contaminants on the matrix. In this procedure, it is general practice to apply alkaline solutions with high concentrations of NaOH such as 0.5 M NaOH to the affinity ligand matrices. Wild-type Protein A domains cannot withstand such harsh alkaline conditions for an extended time and quickly lose binding capacity for immunoglobulin. Further, for a repeated use of affinity ligand matrices, a cleaning step under acidic conditions is usually required.

Accordingly, there is an ongoing need in this field to obtain novel proteins capable of binding proteins comprising an Ig sequence or the Fc region of an Immunoglobulin, for example antibodies, and to better withstand the harsh conditions applied in affinity purification of immunoglobulins.

The present invention provides Ig binding proteins that are particularly well-suited for affinity purification of immunoglobulins. In particular, the Ig binding proteins of the invention have several advantages. One significant advantage of the Ig binding proteins of the invention is their improved stability at high pH (alkaline conditions) for a prolonged time period (such as 1.5 to 2 days at 1 M NaOH) without significantly reducing the Ig binding capacities in combination with high dynamic binding capacities. Further, the novel proteins of the invention are particularly useful for affinity purification of antibodies where mild acidic elution conditions at pH 4.0 or above are required.

The above overview does not necessarily describe all problems solved by the present invention.

### SUMMARY OF THE INVENTION

An aspect of the present invention is to provide an Ig binding protein suitable for affinity purification.

[1] This is achieved with an Immunoglobulin (Ig) binding protein comprising the amino acid sequence of SEQ ID NO: 13, or an Ig binding protein comprising an amino acid sequence with at least 89.5% amino acid identity thereto, wherein the amino acid corresponding to position 5 is phenylalanine (F), the amino acid corresponding to position 8 is isoleucine (I), the amino acid corresponding to position 28 is histidine (H), and the amino acid corresponding to position 42 is lysine (K). In various embodiments, the amino acid corresponding to position 4 is glutamine (Q) or lysine (K), and/or the amino acid corresponding to position 7 is lysine (K) or glutamic acid (E). In various embodiments, the amino acid corresponding to position 58 is proline (P).
[2] The Ig binding protein of item [1], wherein amino acid corresponding to position 9 is alanine (A) or glutamine (Q).
[3] The Ig binding protein of item [1] or [2], wherein the amino acid corresponding to position 11 is alanine (A) or isoleucine (I).
[4] The Ig binding protein of any one of items [1] to [3], wherein the amino acid corresponding to position 15 is alanine (A) or glutamic acid (E).
[5] The Ig binding protein according to any one of items [1] to [4], wherein the Ig binding protein is multimeric and comprises at least 3 Ig binding proteins.
[6] The Ig binding protein according to item [5], wherein the multimeric Ig binding protein is a pentamer.
[7] The Ig binding protein according to any one of items [1] to [6], wherein the Ig binding protein comprises any amino acid sequence with at least 89.5% to any of SEQ ID NOs: 1-18.
[8] The Ig binding protein according to any one of items [1] to [7], wherein said protein binds to one or more of IgG₁, IgG₂, IgG₄, IgM, IgA, Ig fragments, Fc fragments, Fab fragments, fusion proteins comprising an Ig region, and conjugates comprising an Ig region. In various embodiments, the Ig binding protein is binding to proteins comprising an Fc region or is binding to Fc fragments.
In various embodiments, the Ig binding protein is binding to proteins comprising an Fc region or is binding to Fc fragments, optionally with binding affinity of less than 100 nM, as determined via SPR.
[9] The Ig binding protein according to any one of items [1] to [8], wherein the protein is immobilized on a solid support.
[10] The Ig binding protein according to any one of items [1] to [9], wherein the Ig binding protein is stable under alkaline conditions, preferably at 1 M NaOH for at least 37 h, optionally with remaining IgG binding activity of about 80% after incubation at 1 M NaOH for at least 37 h.
[11] An affinity separation matrix comprising the Ig binding protein of any one of items [1] to [10] coupled thereto.
[9] Use of the Ig binding protein of any one of items [1] to [7], or the affinity separation matrix of item [8], for affinity purification, in particular affinity purification of any protein with affinity to the Ig binding protein.
[12] Use of the Ig binding protein of any one of items [1] to [10], or the affinity separation matrix of item [11], for affinity purification, in particular affinity purification of any protein with affinity to the Ig binding protein.
[13] A method for affinity purifying a protein comprising the Fc region of an immunoglobulin (Ig), the method comprising:
a) providing a sample, preferably a liquid sample, that contains a protein comprising the Fc region of an Ig;
b) providing an affinity separation matrix according to item [11];
c) contacting said affinity separation matrix with the (liquid) sample under conditions that permit binding of the at least one Ig binding protein of the affinity separation matrix to the protein comprising the Fc region of an Ig; and
d) collecting, preferably eluting, said protein comprising the Fc region of an Ig from said affinity purification matrix, thereby obtaining the (affinity purified) protein comprising the Fc region of an Ig, preferably obtaining an eluate containing said protein comprising an Fc region of an g.
   [14] The method according to item [13], wherein in step (d) more than about 90%, preferably 95% of the protein comprising the Fc region of an Ig is eluted at pH 4.0 from the affinity separation matrix.
   [15] The method according to any of items [13]-[14], comprising the additional step (e) of washing the affinity purification matrix with an alkaline cleaning liquid, optionally or preferably wherein the Ig binding protein retains at least about 80% Ig binding activity after incubation for at least 37 h at 1 M NaOH.

This summary of the invention does not necessarily describe all features of the present invention. Other embodiments will become apparent from a review of the ensuing detailed description.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which may be limited only by the appended claims. Unless defined otherwise, all technical and scientific terms used herein have the same meanings as commonly understood by one of ordinary skill in the art to which this invention belongs.

Preferably, the terms used herein are consistent with the definitions provided in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

The term “about”, as used herein, encompasses the explicitly recited amounts as well as deviations therefrom of ±10%. More preferably, a deviation of 5% is encompassed by the term “about”.

Several documents (for example: patents, patent applications, scientific publications, manufacturer's specifications etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

All sequences referred to herein are disclosed in the attached sequence listing that, with its whole content and disclosure, is a part of this specification.

In the context of the present invention, the term “Ig binding protein” or “immunoglobulin-binding protein” is used to describe proteins that are capable to specifically bind to an immunoglobulin. Further, in the context of the present invention, the term “Ig binding domain” or “immunoglobulin-binding domain” is used to describe proteins that are capable to specifically bind to an immunoglobulin. The Ig binding proteins or Ig binding domains of the present invention are sometimes referred to herein as ligands of the invention. The “immunoglobulin” or “Ig” as understood herein can include, but is not necessarily limited to, mammalian IgG, such as for example human IgG₁, human IgG₂, human IgG₄, mouse IgG, rat IgG, goat IgG, bovine IgG, guinea pig IgG, rabbit IgG; human IgM, human IgA; and an immunoglobulin or an immunoglobulin fragment comprising a Fc region (also referred to as “Fc fragment” or “Fc”) and/or an immunoglobulin fragment comprising a Fab region (also referred to as “Fab fragment” or “Fab”). The Ig binding proteins are capable of binding to entire immunoglobulins, and to Ig fragments comprising a Fc region and/or Ig fragments comprising a Fab region. The definition “immunoglobulin” as understood herein includes fusion proteins comprising an immunoglobulin, fragment of an immunoglobulin comprising a Fc region (Fc fragment), fragment of an immunoglobulin comprising a Fab region (Fab fragment), fusion proteins comprising a fragment of an immunoglobulin comprising a Fc region, fusion proteins comprising a fragment of an immunoglobulin comprising a Fab region, conjugates comprising an Ig or an Ig fragment comprising a Fc region (Fc fragment), and conjugates comprising an Ig fragment comprising a Fab region (Fab fragment).

As will be appreciated by a person of ordinary skill in the art, the terms “immunoglobulin” and “antibody” may be used interchangeably herein. Any definitions disclosed herein concerning the term “immunoglobulin” apply to the term “antibody” accordingly.

The term “binding” according to the invention preferably relates to a specific binding. “Specific binding” means that an Ig binding protein or an Ig binding domain binds stronger to an immunoglobulin for which it is specific compared to the binding to another non-immunoglobulin target.

The term “binding activity” refers to the ability of an Ig binding protein or Ig binding domain of the invention to bind to immunoglobulin. For example, the binding activity can be determined before and/or after alkaline treatment. The terms (immunoglobulin) “binding activity” and “binding capacity” may be used interchangeably herein. The binding activity can be determined for an Ig binding protein or for an Ig binding protein coupled to a matrix, i.e., for an immobilized Ig binding protein. Also, the binding activity can be determined for an Ig binding domain or for an Ig binding domain coupled to a matrix, i.e., for an immobilized Ig binding domain. The term “artificial” refers to an object that is not naturally occurring, i.e. the term refers to an object that has been produced or modified by man. For example, a polypeptide or polynucleotide sequence that has been generated by man (e.g. for example in a laboratory by genetic engineering, by shuffling methods, or by chemical reactions, etc.) or intentionally modified is artificial.

The term “dissociation constant” or “K_{D}” defines the specific binding affinity. As used herein, the term “K_{D}” (usually measured in “mol/L”, sometimes abbreviated as “M”) is intended to refer to the dissociation equilibrium constant of the particular interaction between a first protein and a second protein. In the context of the present invention, the term K_{D} is particularly used to describe the binding affinity between an Ig binding protein or an Ig binding domain and an immunoglobulin. An Ig binding protein or Ig binding domain of the invention is considered to bind to an immunoglobulin, if it has a dissociation constant K_{D} to immunoglobulin of at least 500 nM or less, or preferably 100 nM or less, more preferably 50 nM or less, even more preferably 10 nM or less.

The terms “protein” and “polypeptide” refer to any linear molecular chain of two or more amino acids linked by peptide bonds and does not refer to a specific length of the product. Thus, “peptides”, “protein”, “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-translational modifications of the polypeptide, including without limitation glycosylation, acetylation, phosphorylation, amidation, proteolytic cleavage, modification by non-naturally occurring amino acids and similar modifications which are well-known in the art. Thus, Ig binding proteins comprising two or more protein domains also fall under the definition of the term “protein” or “polypeptides”.

The terms “alkaline stable” or “alkaline stability” or “caustic stable” or “caustic stability” (also abbreviated as “cs” herein) may be used interchangeably herein and refer to the ability of the Ig binding protein or Ig binding domain of the invention to withstand alkaline conditions without significantly losing the ability to bind to immunoglobulins. The skilled person in this field can easily test alkaline stability by incubating an Ig binding protein or Ig binding domain with, for example, sodium hydroxide solutions, e.g., as described in the Examples, and subsequent testing of the binding capacity or binding activity to immunoglobulin by routine experiments known to someone skilled in the art, for example, by chromatographic approaches. The alkaline stability may be determined by coupling the Ig binding protein or Ig binding domain of the invention to a surface plasmon resonance (SPR) sensor chip, and assaying the binding capacity or binding activity for immunoglobulin before and after exposure to an alkaline solution. The high alkaline treatment can be performed, for instance, in 1 M NaOH for an extended period of time, e.g., at least 30 h, in various embodiments at least 36 h, or even 48 h. Ig binding proteins or Ig binding domains of the invention as well as matrices comprising Ig binding proteins or Ig binding domains of the invention exhibit an “increased” or “improved” alkaline stability, meaning that the molecules and matrices incorporating said Ig binding proteins or Ig binding domains are stable under alkaline conditions for an extended period of time relative to a reference. In various embodiments, the reference may be Ig binding proteins or Ig binding domains, or matrices comprising Ig binding proteins or Ig binding domains, which do not carry the specific amino acid residues at positions 5, 8, 28, and 42, and/or do not carry the specific amino acid substitutions at positions 9, 11, and 15 described elsewhere herein, and/or do not carry the specific amino acid substitutions at positions 4 and/or 7 described elsewhere herein. In various embodiments, the reference Ig binding proteins or Ig binding domains, or matrices comprising Ig binding proteins or Ig binding domains do not carry the specific amino acid substitution at the position corresponding to position 58 (K to P) of SEQ ID NO: 19.

The term “variant” as used herein includes an amino acid sequence of an Ig binding protein or Ig binding domain that differs from another amino acid sequence by at least one amino acid substitution, deletion or insertion. These modifications may be generated by genetic engineering or by chemical synthesis or chemical reactions carried out by man.

The term “conjugate” as used herein relates to a molecule comprising or essentially consisting of at least a first protein attached chemically to other substances such as to a second protein or a non-proteinaceous moiety.

The term “modification” or “amino acid modification” refers to an exchange, a deletion, or an insertion of an amino acid at a particular position in a polypeptide sequence by another amino acid. Given the known genetic code, and recombinant and synthetic DNA techniques, the skilled scientist can readily construct DNAs encoding the amino acid variants.

The term “substitution” or “amino acid substitution” refers to an exchange of an amino acid at a particular position in a polypeptide sequence by another amino acid. The term “deletion” or “amino acid deletion” refers to the removal of an amino acid at a particular position in a polypeptide sequence.

The term “insertions” or “amino acid insertion” refers to the addition of amino acids to the polypeptide sequence.

Throughout this description, the amino acid residue position numbers are designated as corresponding to those for example in SEQ ID NO: 1. Accordingly, for example, an amino acid corresponding to position 5 means an amino acid residue at the position corresponding to position 5 of SEQ ID NO: 1. In embodiments of the present invention pertaining to SEQ ID NOs: 1-6, 13, 15, 17 and 19, the amino acid corresponding to position 5 is phenylalanine (F), the amino acid corresponding to position 8 is isoleucine (1), the amino acid corresponding to position 28 is histidine (H), and the amino acid corresponding to position 42 is lysine (K). Further, in embodiments of the present invention pertaining to SEQ ID NOs: 1-6, 13, 15, 17 and 19, the amino acid corresponding to position 9 is alanine (A) or glutamine (Q), the amino acid corresponding to position 11 is alanine (A) or isoleucine (1), and/or the amino acid corresponding to position 15 is alanine (A) or glutamic acid (E). Further, in embodiments of the present invention pertaining to SEQ ID NOs: 1, 2, 3, 4, 5, 6, 13, 15, 17, and 19, the amino acid corresponding to position 9 is alanine (A) or glutamine (Q), the amino acid corresponding to position 11 is alanine (A) or isoleucine (1), the amino acid corresponding to position 15 is alanine (A) or glutamic acid (E), the amino acid corresponding to position 4 is glutamine (Q) or lysine (K), and/or the amino acid corresponding to position 7 is lysine (K) or glutamic acid (E). With regard to the corresponding positions in multimers of the invention, in particular pentamers, reference is made to Table 1 below. The term “amino acid sequence identity” refers to a quantitative comparison of the identity (or differences) of the amino acid sequences of two or more proteins. “Percent (%) amino acid sequence identity” or “percent identical” or ““percent identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. In various embodiments, the term “sequence identity” means that two (nucleotide or) amino acid sequences, when optimally aligned, such as by the programs GAP or BESTFIT using default gap weights, share at least 85% sequence identity, or at least 89.5% sequence identity, or at least 91% sequence identity, or at least 95% sequence identity or more.

To determine the sequence identity, the sequence of a query protein is aligned and compared to the sequence of a reference protein. Methods for sequence alignment and sequence comparison algorithms are well known in the art. For example, for determining the extent of an amino acid sequence identity of an arbitrary polypeptide relative to the reference amino acid sequence, the SIM Local similarity program is preferably employed. For multiple alignment analysis, ClustalW as known to someone skilled in the art is preferably used.

The extent of sequence identity is generally calculated with respect to the total length of the unmodified sequence. As used herein, the phrases “percent identical” or “percent (%) amino acid sequence identity” or “percent identity”, in the context of two polypeptide sequences, refer to two or more sequences or subsequences that have in some embodiments at least 89.5%, in some embodiments at least 91%, some embodiments at least 92%, in some embodiments at least 93%, in some embodiments at least 94%, in some embodiments at least 95%, in some embodiments at least 96%, in some embodiments at least 97%, in some embodiments at least 98%, and in some embodiments 100% amino acid residue identity, when compared and aligned for maximum correspondence, as measured using one of the following sequence comparison algorithms or by visual inspection. For clarity reasons, for example a sequence with at least 89.5% identity includes all sequences with identities higher than 89.5% identity, e.g. embodiments with at least 89.6%, at least 90%, at least 91%, at least 92%, at least 93%, at least 94%, at least 95%, at least 96%, at least 97%, at least 98%, at least 99%, or 100% amino acid identity.

The percent identity exists in some embodiments over a region of at least 52 residues, in some embodiments over a region of at least 53 residues, in some embodiments over a region of at least 54 residues, in some embodiments over a region of at least 55 residues, in some embodiments over a region of at least 56 residues, in some embodiments over a region of at least 57 residues, and in some embodiments over a region of at least 58 residues. In certain embodiments, the percent identity exists over a region of (at least) 56 residues, e.g. with regard to an Ig binding protein described herein having a deletion of amino acid positions corresponding to positions 1 and 2 of any of SEQ ID NOs: 1-6, 13, 15, 17, and 19.

The term “fused” means that polypeptide components or units are linked by peptide bonds, either directly or via peptide linkers. In various embodiments, the term “fused” may mean that polypeptide components or units are linked by a non-peptide linker, e.g., through chemical conjugation.

The term “fusion protein” relates to a protein comprising at least a first protein joined genetically to at least a second protein. A fusion protein is created through joining of two or more genes that originally coded for separate proteins. Thus, a fusion protein may comprise a multimer of identical or different proteins which are expressed as a single, linear polypeptide. In various embodiments, a fusion protein is created through joining of two or more polypeptides via a non-peptide linker, e.g., through chemical conjugation. In various embodiments, a dimer of an Ig binding protein or Ig binding domain of the present invention may be considered as a “fusion protein”.

As used herein, the term “linker” refers in its broadest meaning to a molecule that covalently joins at least two other molecules. In typical embodiments of the present invention, a “linker” is to be understood as a moiety that connects an Ig binding protein or Ig binding domain with at least one further Ig binding protein or Ig binding domain, i.e. a moiety linking two protein domains to each other to generate a dimer or a multimer. In preferred embodiments, the “linker” is a peptide linker, i.e. the moiety linking the two binding proteins or binding domains is one single amino acid or a peptide comprising two or more amino acids. In various embodiments, a dimer or multimer of the present invention may comprise a linker joining two or more Ig binding proteins or Ig binding domains with each other.

The term “chromatography” refers to separation technologies which employ a mobile phase and a stationary phase to separate one type of molecules (e.g., immunoglobulins) from other molecules (e.g. contaminants or other immunoglobulins) in the sample. The liquid mobile phase contains a mixture of molecules and transports these across or through a stationary phase (such as a solid matrix). Due to the differential interaction of the different molecules in the mobile phase with the stationary phase, molecules in the mobile phase can be separated. The term “affinity chromatography” refers to a specific mode of chromatography in which a ligand coupled to a stationary phase interacts with a molecule (i.e. immunoglobulin) in the mobile phase (the sample) i.e. the ligand has a specific binding affinity or binding capacity for the molecule to be purified. As understood in the context of the invention, affinity chromatography involves the addition of a (liquid) sample containing an immunoglobulin to a stationary phase which comprises a chromatography ligand, such as an Ig binding protein or Ig binding domain of the invention.

The terms “solid support” or “solid matrix” are used interchangeably herein, and in various embodiments are used for the stationary phase.

The terms “affinity matrix” or “affinity separation matrix” or “affinity chromatography matrix”, as used interchangeably herein, refer to a matrix, e.g. a chromatographic matrix, onto which an affinity ligand e.g., an Ig binding protein or Ig binding domain of the invention is attached. The ligand (e.g., Ig binding protein or Ig binding domain) is capable of specific binding to a molecule of interest (e.g., an immunoglobulin as defined above) which is to be purified or removed from a mixture (in a liquid sample). As will be appreciated by a person of ordinary skill in the art, the terms “affinity matrix” or “affinity separation matrix” or “affinity chromatography matrix” describe the separation of a molecule of interest (in particular an immunoglobulin) by using an Ig binding protein or Ig binding domain of the invention. Accordingly, the terms “affinity matrix” or “affinity separation matrix” or “affinity chromatography matrix” or “separation matrix” may be used interchangeably herein.

The term “affinity purification” as used herein refers to a method of purifying immunoglobulins of interest as defined above from a liquid (sample) by binding immunoglobulins of interest as defined above to an Ig binding protein or Ig binding domain that is immobilized to a matrix. Thereby, all other components of the mixture except immunoglobulins of interest are removed. In various embodiments, said other components of the mixture may include, e.g., other immunoglobulins that are not of interest. In a further step, immunoglobulins of interest are eluted in purified form. The terms “affinity purification” or “affinity chromatography purification” or “affinity separation” or “affinity chromatography separation” may be used interchangeably herein.

### EMBODIMENTS OF THE INVENTION

The present invention will now be further described. In the following passages different embodiments of the invention are defined in more detail. Each embodiment defined below may be combined with any other embodiments unless clearly indicated to the contrary. In particular, any feature indicated as being preferred or advantageous may be combined with any other feature or features indicated as being preferred or advantageous.

The present invention provides an Immunoglobulin (Ig) binding protein comprising an amino acid sequence of SEQ ID NO: 13, or an Ig binding protein comprising an amino acid sequence with at least 89.5% amino acid identity thereto wherein the amino acid corresponding to position 5 of SEQ ID NO: 13 is phenylalanine (F), the amino acid corresponding to position 8 of SEQ ID NO: 13 is isoleucine (I), the amino acid corresponding to position 28 of SEQ ID NO: 13 is histidine (H), and the amino acid corresponding to position 42 of SEQ ID NO: 13 is lysine (K).

In various embodiments, the amino acid corresponding to position 9 of SEQ ID NO: 13 is alanine (A) or glutamine (Q).

In various embodiments, the amino acid corresponding to position 11 of SEQ ID NO: 13 is alanine (A) or isoleucine (I).

In various embodiments, the amino acid corresponding to position 15 of SEQ ID NO: 13 is alanine (A) or glutamic acid (E).

In various embodiments, the amino acid corresponding to position 4 of SEQ ID NO: 13 is lysine (K) or glutamine (Q).

In various embodiments, the amino acid corresponding to position 7 of SEQ ID NO: 13 is lysine (K) or glutamic acid (E).

In various embodiments, the amino acid corresponding to position 58 of SEQ ID NO: 13 is lysine (K) or proline (P).

In some embodiments, the Ig binding protein or Ig binding domain of the invention is selected from the following (1) to (2):
(1) a protein comprising an amino acid sequence corresponding to SEQ ID NO: 13;
(2) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 13 specified in the (1) is not mutated.

In some embodiments, the Ig binding protein or Ig binding domain of the invention is selected from the following (1) to (6):
(1) a protein comprising an amino acid sequence corresponding to SEQ ID NO: 1 or SEQ ID NO: 2 or SEQ ID NO: 3 or SEQ ID NO: 4 or SEQ ID NO: 5 or SEQ ID NO: 6 or SEQ ID NO: 17 or SEQ ID NO: 19, wherein an amino acid residue corresponding to the 5^{th} position is Phe (F) and an amino acid residue corresponding to the 8th position is lie (I) and an amino acid residue corresponding to the 28^{th} position is His (H) and an amino acid residue corresponding to the 42^{nd} position is Lys (K);
(2) a protein comprising an amino acid sequence having a sequence identity of at least 89.5% or more with the amino acid sequence specified in the (1), provided that the amino acid at the 5^{th}, 8^{th}, 28^{th} and 42^{nd} position of any one of SEQ ID NOs: 1, 2, 3, 4, 5, 6, 17, or 19, specified in the (1) is not mutated;
(3) a protein comprising an amino acid sequence having a sequence identity of at least 89.5% or more with the amino acid sequence specified in the (1), wherein the amino acid corresponding to position 4 of any one of SEQ ID NO: 1, 2, 3, 4, 5, 6 or 17, is lysine (K), and the amino acid corresponding to position 7 of any one of SEQ ID NO: 1, 2, 3, 4, 5, 6 or 17, is glutamic acid (E);
(4) a protein comprising an amino acid sequence having a sequence identity of at least 89.5% or more with the amino acid sequence specified in the (1), wherein the amino acid corresponding to position 4 of any one of SEQ ID NO: 1, 2, 3, 4, 5, 6, 17, or 19, is glutamine (Q), and the amino acid corresponding to position 7 of any one of SEQ ID NO: 1, 2, 3, 4, 5, 6 or 17, is lysine (K);
(5) a protein comprising an amino acid sequence having a sequence identity of at least 89.5% or more with the amino acid sequence specified in the (1), wherein the amino acid corresponding to position 4 of any one of SEQ ID NO: 1, 2, 3, 4, 5, 6 or 17, is glutamine (Q), and the amino acid corresponding to position 7 of any one of SEQ ID NO: 1, 2, 3, 4, 5, 6 or 17, is glutamic acid (E);
(6) a protein comprising an amino acid sequence having a sequence identity of at least 89.5% or more with the amino acid sequence specified in the (1), wherein the amino acid corresponding to position 4 of any one of SEQ ID NO: 1, 2, 3, 4, 5, 6 or 17, is lysine (K), and the amino acid corresponding to position 7 of any one of SEQ ID NO: 1, 2, 3, 4, 5, 6 or 17, is lysine (K).

In some embodiments, the Ig binding protein or Ig binding domain of the invention is selected from the following (1) to (4):
(1) a protein comprising an amino acid sequence of SEQ ID NO: 13;
(2) a protein comprising an amino acid sequence specified in the (1) wherein the amino acid residue corresponding to position 9 of SEQ ID NO: 13 is Ala (A) or Gln (Q), and/or wherein the amino acid residue corresponding to the position 11 of SEQ ID NO: 13 is lie (1) or Ala (A), and/or wherein amino acid residue corresponding to the position 15 of SEQ ID NO: 13 is Glu (E) or Ala (A);
(3) a protein comprising an amino acid sequence specified in the (1) wherein the amino acid residue corresponding to position 4 of SEQ ID NO: 13 is lysine (K) or glutamine (Q) and/or wherein the amino acid residue corresponding to position 7 of SEQ ID NO: 13 is lysine (K) or glutamic acid (E), and/or wherein the amino acid residue corresponding to position 9 of SEQ ID NO: 13 is Ala (A) or Gln (Q), and/or wherein the amino acid residue corresponding to the position 11 of SEQ ID NO: 13 is lie (I) or Ala (A), and/or wherein amino acid residue corresponding to the position 15 of SEQ ID NO: 13 is Glu (E) or Ala (A); (4) a protein comprising an amino acid sequence having a sequence identity of at least 89.5% or more with the amino acid sequence specified in the (1), provided that the amino acid specified in the (1) at the 5^{th}, 8^{th}, 28^{th} and 42^{nd} position is not mutated.

The present invention further provides an Immunoglobulin (Ig) binding protein comprising an amino acid sequence of SEQ ID NO: 15, or an Ig binding protein comprising an amino acid sequence with at least 89.5% amino acid identity thereto, wherein the amino acid corresponding to position 5 of SEQ ID NO: 15 is phenylalanine (F), the amino acid corresponding to position 8 of SEQ ID NO: 15 is isoleucine (I), the amino acid corresponding to position 28 of SEQ ID NO: 15 is histidine (H), the amino acid corresponding to position 42 of SEQ ID NO: 15 is lysine (K). In various preferred embodiments, the amino acid corresponding to position 4 of SEQ ID NO: 15 is lysine (K), and the amino acid corresponding to position 7 of SEQ ID NO: 15 is glutamic acid (E). In various other embodiments, the amino acid corresponding to position 4 of SEQ ID NO: 15 is glutamine (Q), and the amino acid corresponding to position 7 of SEQ ID NO: 15 is lysine (K). In various further embodiments, the amino acid corresponding to position 4 of SEQ ID NO: 15 is glutamine (Q), and the amino acid corresponding to position 7 of SEQ ID NO: 15 is glutamic acid (E). Still further, in various embodiments, the amino acid corresponding to position 4 of SEQ ID NO: 15 is lysine (K), and the amino acid corresponding to position 7 of SEQ ID NO: 15 is lysine (K). In various further embodiments, the amino acid corresponding to position 58 of SEQ ID NO: 15 is proline (P). In various further embodiments, the amino acid corresponding to position 4 of SEQ ID NO: 15 is glutamine (Q), the amino acid corresponding to position 7 of SEQ ID NO: 15 is lysine (K), the amino acid corresponding to positions 9 and 11 of SEQ ID NO: 15 is alanine (A), the amino acid corresponding to position 15 of SEQ ID NO: 15 is glutamic acid (E), and the amino acid corresponding to position 58 of SEQ ID NO: 15 is proline (P).

In some embodiments, the Ig binding protein or Ig binding domain of the invention is selected from the following (1) to (4):
(1) a protein comprising an amino acid sequence corresponding to SEQ ID NO: 15;
(2) a protein comprising an amino acid sequence having a sequence identity of at least 89.5% or more with the amino acid sequence specified in the (1), provided that the amino acids at the 5^{th}, 8^{th}, 28^{th} and 42^{nd} position of SEQ ID NO: 15 specified in the (1) are not mutated.
(3) a protein comprising an amino acid sequence having a sequence identity of at least 89.5% or more with the amino acid sequence specified in the (1), provided that the amino acids corresponding to the amino acids at the 5^{th}, 8^{th}, 28^{th}, and 42^{nd} position of SEQ ID NO: 15 specified in the (1) are not mutated, and the amino acid corresponding to the amino acid at position 9 of SEQ ID NO: 15 is Ala (A) or Gln (Q), and/or the amino acid corresponding to the amino acid at position 11 of SEQ ID NO: 15 is lie (I) or Ala (A), and/or the amino acid corresponding to the amino acid at position 15 of SEQ ID NO: 15 is Glu (E) or Ala (A). (4) a protein comprising an amino acid sequence having a sequence identity of at least 89.5% or more with the amino acid sequence specified in the (1), provided that the amino acids corresponding to the amino acids at the 5^{th}, 8^{th}, 28^{th} and 42^{nd} position of SEQ ID NO: 15 specified in the (1) are not mutated, and the amino acid corresponding to the amino acid at position 9 of SEQ ID NO: 15 is Ala (A) or Gln (Q), and/or the amino acid corresponding to the amino acid at position 11 of SEQ ID NO: 15 is lie (I) or Ala (A), and/or the amino acid corresponding to the amino acid at position 15 of SEQ ID NO: 15 is Glu (E) or Ala (A), and wherein the amino acid corresponding to the amino acid at position 4 of SEQ ID NO: 15 is lysine (K) or glutamine (Q) and/or the amino acid corresponding to the amino acid at position position 7 of SEQ ID NO: 15 is lysine (K) or glutamic acid (E); in various preferred embodiments, the amino acid corresponding to position 4 of SEQ ID NO: 15 is lysine (K), and the amino acid corresponding to position 7 of SEQ ID NO: 15 is glutamic acid (E); in various other embodiments, the amino acid corresponding to position 4 of SEQ ID NO: 15 is glutamine (Q), and the amino acid corresponding to position 7 of SEQ ID NO: 15 is lysine (K); in various further embodiments, the amino acid corresponding to position 4 of SEQ ID NO: 15 is glutamine (Q), and the amino acid corresponding to position 7 of SEQ ID NO: 15 is glutamic acid (E); still further, in various embodiments, the amino acid corresponding to position 4 of SEQ ID NO: 15 is lysine (K), and the amino acid corresponding to position 7 of SEQ ID NO: 15 is lysine (K).

In some embodiments, the Ig binding protein or Ig binding domain of the invention is selected from the following (1) to (6):
(1) a protein comprising an amino acid sequence corresponding to SEQ ID NO: 1;
(2) a protein comprising an amino acid sequence specified in the (1) further having a substitution of one or more amino acid residues in a position other than the 5^{th}, 8^{th}, 28^{th} and 42^{nd} position, preferably having a substitution of one or more amino acid residues in any of the 30 the 4^{th}, 7^{th}, 9^{th}, 11^{th}, and 15^{th} position of SEQ ID NO: 1 specified in the (1);
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 1 specified in the (1) is not mutated.
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 1 specified in the (1) is not mutated, and the amino acid at the 9^{th}, 11^{th}, and 15^{th} position of SEQ ID NO: 1 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 1 specified in the (1) is not mutated, and the amino acid at the 9^{th}, 11^{th}, and 15^{th} position of SEQ ID NO: 1 specified in the (1) is not mutated; (6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 1 specified in the (1) is not mutated, and the amino acid at the 9^{th}, 11^{th}, and 15^{th} position of SEQ ID NO: 1 specified in the (1) is not mutated, and the amino acid at the 4^{th} and 7^{th} position of SEQ ID NO: 1 specified in the (1) is not mutated.

In some embodiments, the Ig binding protein or Ig binding domain of the invention is selected from the following (1) to (6):
(1) a protein comprising an amino acid sequence corresponding to SEQ ID NO: 2;
(2) a protein comprising an amino acid sequence specified in the (1) further having a substitution of one or more amino acid residues in one or more positions other than the 5^{th}, 8^{th}, 28^{th}, and 42^{nd} position of SEQ ID NO: 2, preferably having a substitution of one or more amino acid residues in any one of the 4^{th}, 7^{th}, 9^{th}, 11^{th}, and 15^{th} position of SEQ ID NO: 2 specified in the (1);
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 2 specified in the (1) is not mutated;
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 2 specified in the (1) is not mutated, and the amino acid at the 9^{th}, 11^{th}, and 15^{th} position of SEQ ID NO: 2 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 2 specified in the (1) is not mutated, and the amino acid at the 9^{th}, 11^{th}, and 15^{th} position of SEQ ID NO: 2 specified in the (1) is not mutated;
(6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 2 specified in the (1) is not mutated, and the amino acid at the 9^{th}, 11^{th}, and 15^{th} position of SEQ ID NO: 2 specified in the (1) is not mutated, and the amino acid at the 4^{th} and 7^{th} position of SEQ ID NO: 2 specified in the (1) is not mutated.

In some embodiments, the Ig binding protein or Ig binding domain of the invention is selected from the following (1) to (6):
(1) a protein comprising an amino acid sequence corresponding to SEQ ID NO: 3;
(2) a protein comprising an amino acid sequence specified in the (1) further having a substitution of one or more amino acid residues in one or more positions other than the 5^{th}, 8^{th}, 28^{th}, and 42^{nd} position of SEQ ID NO: 3, preferably having a substitution of one or more amino acid residues in any one of the 4^{th}, 7^{th}, 9^{th}, 11^{th}, and 15^{th} position of SEQ ID NO: 3 specified in the (1);
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 3 specified in the (1) is not mutated;
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 3 specified in the (1) is not mutated, and the amino acid at the 9^{th}, 11^{th}, and 15^{th} position of SEQ ID NO: 3 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 3 specified in the (1) is not mutated, and the amino acid at the 9^{th}, 11^{th}, and 15^{th} position of SEQ ID NO: 3 specified in the (1) is not mutated;
(6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 3 specified in the (1) is not mutated, and the amino acid at the 9^{th}, 11^{th}, and 15^{th} position of SEQ ID NO: 3 specified in the (1) is not mutated, and the amino acid at the 4^{th} and 7^{th} position of SEQ ID NO: 3 specified in the (1) is not mutated.

In various embodiments pertaining to SEQ ID NO: 3 described under the above (1) and (6), and described elsewhere herein, the amino acid corresponding to position 58 of SEQ ID NO: 15 is proline (P).

In some embodiments, the Ig binding protein or Ig binding domain of the invention is selected from the following (1) to (6):
(1) a protein comprising an amino acid sequence corresponding to SEQ ID NO: 4;
(2) a protein comprising an amino acid sequence specified in the (1) further having a substitution of one or more amino acid residues in one or more positions other than the 5^{th}, 8^{th}, 28^{th}, and 42^{nd} position of SEQ ID NO: 4, preferably having a substitution of one or more amino acid residues in any one of the 4^{th}, 7^{th}, 9^{th}, 11^{th}, and 15^{th} position of SEQ ID NO: 4 specified in the (1);
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 4 specified in the (1) is not mutated;
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 4 specified in the (1) is not mutated, and the amino acid at the 9^{th}, 11^{th}, and 15^{th} position of SEQ ID NO: 4 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 4 specified in the (1) is not mutated, and the amino acid at the 9^{th}, 11^{th}, and 15^{th} position of SEQ ID NO: 4 specified in the (1) is not mutated;
(6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 4 specified in the (1) is not mutated, and the amino acid at the 9^{th}, 11^{th}, and 15^{th} position of SEQ ID NO: 4 specified in the (1) is not mutated, and the amino acid at the 4^{th} and 7^{th} position of SEQ ID NO: 4 specified in the (1) is not mutated.

In some embodiments, the Ig binding protein or Ig binding domain of the invention is selected from the following (1) to (6):
(1) a protein comprising an amino acid sequence corresponding to SEQ ID NO: 5;
(2) a protein comprising an amino acid sequence specified in the (1) further having a substitution of one or more amino acid residues in one or more positions other than the 5^{th}, 8^{th}, 28^{th}, and 42^{nd} position of SEQ ID NO: 5, preferably having a substitution of one or more amino acid residues in any one of the 4^{th}, 7^{th}, 9^{th}, 11^{th}, and 15^{th} position of SEQ ID NO: 5 specified in the (1);
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 5 specified in the (1) is not mutated.
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 5 specified in the (1) is not mutated, and the amino acid at the 9^{th}, 11^{th}, and 15^{th} position of SEQ ID NO: 5 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 5 specified in the (1) is not mutated, and the amino acid at the 9^{th}, 11^{th}, and 15^{th} position of SEQ ID NO: 5 specified in the (1) is not mutated;
(6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 5 specified in the (1) is not mutated, and the amino acid at the 9^{th}, 11^{th}, and 15^{th} position of SEQ ID NO: 5 specified in the (1) is not mutated, and the amino acid at the 4^{th} and 7^{th} position of SEQ ID NO: 5 specified in the (1) is not mutated.

In some embodiments, the Ig binding protein or Ig binding domain of the invention is selected from the following (1) to (6):
(1) a protein comprising an amino acid sequence corresponding to SEQ ID NO: 6;
(2) a protein comprising an amino acid sequence specified in the (1) further having a substitution of one or more amino acid residues in one or more positions other than the 5^{th}, 8^{th}, 28^{th}, and 42^{nd} position of SEQ ID NO: 6, preferably having a substitution of one or more amino acid residues in any one of the 4^{th}, 7^{th}, 9^{th}, 11^{th}, and 15^{th} position of SEQ ID NO: 6 specified in the (1);
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 6 specified in the (1) is not mutated;
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 6 specified in the (1) is not mutated, and the amino acid at the 9^{th}, 11^{th}, and 15^{th} position of SEQ ID NO: 6 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 6 specified in the (1) is not mutated, and the amino acid at the 9^{th}, 11^{th}, and 15^{th} position of SEQ ID NO: 6 specified in the (1) is not mutated;
(6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 6 specified in the (1) is not mutated, and the amino acid at the 9^{th}, 11^{th}, and 15^{th} position of SEQ ID NO: 6 specified in the (1) is not mutated, and the amino acid at the 4^{th} and 7^{th} position of SEQ ID NO: 6 specified in the (1) is not mutated.

In some embodiments, the Ig binding protein or Ig binding domain of the invention is selected from the following (1) to (6):
(1) a protein comprising an amino acid sequence corresponding to SEQ ID NO: 17;
(2) a protein comprising an amino acid sequence specified in the (1) further having a substitution of one or more amino acid residues in one or more positions other than the 5^{th}, 8^{th}, 28^{th}, and 42^{nd} position of SEQ ID NO: 17, preferably having a substitution of one or more amino acid residues in any one of the 4^{th}, 7^{th}, 9^{th}, 11^{th}, and 15^{th} position of SEQ ID NO: 17;
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 17 specified in the (1) is not mutated;
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 17 specified in the (1) is not mutated, and the amino acid at the 9^{th}, 11^{th}, and 15^{th} position of SEQ ID NO: 17 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 17 specified in the (1) is not mutated, and the amino acid at the 9^{th}, 11^{th}, and 15^{th} position of SEQ ID NO: 17 specified in the (1) is not mutated;
(6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 17 specified in the (1) is not mutated, and the amino acid at the 9^{th}, 11^{th}, and 15^{th} position of SEQ ID NO: 17 specified in the (1) is not mutated, and the amino acid at the 4^{th} and 7^{th} position of SEQ ID NO: 17 specified in the (1) is not mutated.

The present invention encompasses an Immunoglobulin (Ig) binding protein comprising the amino acid sequence of SEQ ID NO: 19, or an Ig binding protein comprising an amino acid sequence with at least 89.5% amino acid identity thereto, wherein the amino acid corresponding to position 5 of SEQ ID NO: 19 is phenylalanine (F), the amino acid corresponding to position 8 of SEQ ID NO: 19 is isoleucine (I), the amino acid corresponding to position 28 of SEQ ID NO: 19 is histidine (H), and the amino acid corresponding to position 42 of SEQ ID NO: 19 is lysine (K). In preferred embodiments pertaining to SEQ ID NO: 19, the amino acid corresponding to position 5 is F, the amino acid corresponding to position 8 is I, the amino acid corresponding to position 28 is H, the amino acid corresponding to position 42 is K, the amino acid corresponding to position 4 is glutamine (Q), the amino acid corresponding to position 7 is lysine (K), the amino acid corresponding to positions 9 and 11 is alanine (A), the amino acid corresponding to position 15 is glutamic acid (E), and the amino acid corresponding to position 58 is proline (P).

In some embodiments, the Ig binding protein or Ig binding domain of the invention is selected from the following (1) to (7):
(1) a protein comprising an amino acid sequence corresponding to SEQ ID NO: 19;
(2) a protein comprising an amino acid sequence specified in the (1) further having a substitution of one or more amino acid residues in one or more positions other than the 5^{th}, 8^{th}, 28^{th}, and 42^{nd} position of SEQ ID NO: 19, preferably having a substitution of one or more amino acid residues in any one of the 4^{th}, 7^{th}, 9^{th}, 11^{th}, and 15^{th} position of SEQ ID NO: 19;
(3) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 19 specified in the (1) is not mutated;
(4) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 19 specified in the (1) is not mutated, and the amino acid at the 9^{th}, 11^{th}, and 15^{th} position of SEQ ID NO: 19 specified in the (1) is not mutated;
(5) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 19 specified in the (1) is not mutated, and the amino acid at the 9^{th}, 11^{th}, and 15^{th} position of SEQ ID NO: 19 specified in the (1) is not mutated;
(6) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 19 specified in the (1) is not mutated, and the amino acid at the 9^{th}, 11^{th}, and 15^{th} position of SEQ ID NO: 19 specified in the (1) is not mutated, and the amino acid at the 4^{th} and 7^{th} position of SEQ ID NO: 19 specified in the (1) is not mutated;
(7) a protein comprising an amino acid sequence having a sequence identity of at least 89.5% or more with the amino acid sequence specified in the (1), provided that the amino acid at the 5^{th}, 8^{th}, 28^{th} and 42^{nd} position of SEQ ID NO: 19 specified in the (1) is not mutated, the amino acid at the 9^{th}, 11^{th}, and 15^{th} position of SEQ ID NO: 19 specified in the (1) is not mutated, the amino acid at the 4^{th} and 7^{th} position of SEQ ID NO: 19 specified in the (1) is not mutated, and the amino acid at the 58^{th} position of SEQ ID NO: 19 specified in the (1) is not mutated.

The surprising advantage of the Ig binding proteins and Ig binding domains of the invention is the stability under extreme conditions such as high pH (pH 13 and higher) without losing Ig binding properties. The Ig binding proteins and Ig binding domains as described herein demonstrate high alkali stability for a prolonged period of time (at least 37 hours) without impairing the Ig-binding properties (see Examples). Further, they are stable at low pH without significantly losing Ig binding properties. The extreme high alkali stability feature is particularly important for chromatography approaches with harsh cleaning procedures using alkaline solutions with NaOH concentrations as high as 1 M to remove contaminants on the matrix so that the matrix can be used several times. In addition to extreme high caustic stability, Ig binding proteins show high coupling efficiencies, as shown in the Examples.

Further, an important step in affinity chromatography is the elution of the protein of interest, particular an immunoglobulin of interest, that is bound to the Ig binding protein or Ig binding domain of the invention. This step is usually done at low pH. The affinity ligands of the invention do not lose binding properties to Ig after this treatment, while elution of the protein of interest is possible at low pH. In some circumstances, it is important to have conditions for the elution of antibodies (immunoglobulins) from the ligand at pH higher than pH 3.7, such as pH 4.0 or pH 4.5, and above. The ligands of the invention have a remaining IgG binding capacity of about 80% after at least 37 hours at 1 M NaOH and elution of at least about 92%, at least about 95%, preferably at least 97%, more preferably 100% of the bound IgG from the matrix at mild pH of 4.0 or higher.

In preferred embodiments of the invention, ligands of the invention showing (or providing for), after incubation of at least 37 hours at 1 M NaOH, an elution of at least about 92% of bound Ig (in particular IgG) from an affinity purification or separation matrix at pH 4.0 (or higher) include Ig binding proteins or Ig binding domains described herein in relation to the monomers of any of SEQ ID NOs: 1-4, 6, 17, 19 (including multimers such as the respective pentamers of the SEQ ID NOs: 7, 8, 9, 10, 12, 18, and 20).

In other preferred embodiments of the invention, ligands of the invention showing (or providing for), after incubation of at least 37 hours at 1 M NaOH, an elution of at least about 94% of bound Ig (in particular IgG) from an affinity purification or separation matrix at pH 4.0 (or higher) specifically include Ig binding proteins or Ig binding domains described herein in relation to the monomer of SEQ ID NO: 6 (including multimers such as the respective pentamer of SEQ ID NO: 12).

In further preferred embodiments of the invention, ligands of the invention showing (or providing for), after incubation of at least 37 hours at 1 M NaOH, an elution of at least about 97% of bound Ig (in particular IgG) from an affinity purification or separation matrix at pH 4.0 (or higher) specifically include Ig binding proteins or Ig binding domains described herein in relation to the monomers of SEQ ID NOs: 2 and 4 (including multimers such as the respective pentamers of SEQ ID NOs: 8 and 10).

Still further, preferred ligands of the invention showing (or providing for), after incubation of at least 37 hours at 1 M NaOH, an elution of about 99%, or even 100%, of bound Ig (in particular IgG) from an affinity purification or separation matrix at pH 4.0 (or higher) specifically include Ig binding proteins or Ig binding domains described herein in relation to the monomers of SEQ ID NOs: 1 and 3 (including multimers such as the respective pentamers of SEQ ID NOs: 7 and 9).

Still further, preferred ligands of the invention showing (or providing for), after incubation of at least 37 hours at 1 M NaOH, an elution of at least about 84% of bound Ig (in particular IgG) from an affinity purification or separation matrix at pH 4.3 (or higher) specifically include Ig binding proteins or Ig binding domains described herein in relation to the monomer of SEQ ID NO: 17 (including multimers such as the respective pentamer of SEQ ID NOs: 18).

Still further, preferred ligands of the invention showing (or providing for), after incubation of at least 37 hours at 1 M NaOH, an elution of at least about 84% of bound Ig (in particular IgG) from an affinity purification or separation matrix at pH 4.5 (or higher) specifically include Ig binding proteins or Ig binding domains described herein in relation to the monomer of SEQ ID NO: 17 (including multimers such as the respective pentamer of SEQ ID NOs: 18).

Further modifications can be introduced to the protein to modify certain properties for affinity chromatography. For example, a cysteine can be added to the C-terminus. Alternatively, a cysteine can be introduced at a position corresponding to position 43 or position 46 (of, e.g., any of SEQ ID NOs: 1-6, 13, 15, 17, and 19), to enable efficient coupling to the matrix.

In some embodiments, the Ig binding protein or Ig binding domain is selected from the following (1) to (2):
(1) protein comprising an amino acid sequence corresponding to an amino acid sequence selected from the group of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19, wherein the amino acid residue corresponding to the 5^{th} position is Phe (F) and the amino acid residue corresponding to the 8th position is lie (I) and the amino acid residue corresponding to the 28^{th} position is His (H), and the amino acid residue corresponding to the 42^{nd} position is Lys (K);
(2) a protein comprising an amino acid sequence having a sequence identity of at least 89.5% or more with the amino acid sequence specified in the (1), provided that the amino acids specified in (1) are not modified in (2).

As described herein, the term “sequence identity of at least 89.5% or more” includes preferred embodiments, in which the sequence identity is at least 90%, at least 91%, at least 92%, at least 93%, at least 94%, at least 95%, at least 96%, at least 97%, at least 98%, or at least 99% identity. Such preferred sequence identities are in accordance with the sequence identities described elsewhere herein in relation to SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19 Preferred Iq binding domains. In various embodiments, the Ig binding protein comprises or consists of an amino acid sequence of any of the Ig binding domains described herein. In various embodiments, the Ig binding protein comprises or consists of an amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19, or an amino acid with at least 89.5%, at least 90%, at least 91%, at least 92%, at least 93%, at least 94%, at least 95%, at least 96%, at least 97%, at least 98%, or at least 99% identity thereto, provided that the amino acid residue corresponding to the 5^{th} position is Phe (F) and the amino acid residue corresponding to the 8th position is lie (I) and the amino acid residue corresponding to the 28^{th} position is His (H), and the amino acid residue corresponding to the 42^{nd} position is Lys (K). In various embodiments, the Ig binding domain comprises or essentially consists of or consists of an amino acid sequence of any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19, or an amino acid with at least 95%, at least 96%, at least 97%, at least 98%, or at least 99% identity to any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19 provided that wherein the amino acid residue corresponding to the 5^{th} position is Phe (F) and the amino acid residue corresponding to the 8th position is lie (I) and the amino acid residue corresponding to the 28^{th} position is His (H), and the amino acid residue corresponding to the 42^{nd} position is Lys (K).

Preferred Iq binding proteins. In some embodiments, the Ig binding protein comprises one or more binding domains wherein at least one domain comprises or consists of an amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19 or an amino acid with at least 89.5%, at least 90%, at least 91%, at least 92%, at least 93%, at least 94%, at least 95%, at least 96%, at least 97%, at least 98%, or at least 99% identity thereto, wherein the amino acid residue corresponding to the 5^{th} position is Phe (F) and the amino acid residue corresponding to the 8th position is lie (I) and the amino acid residue corresponding to the 28^{th} position is His (H), and the amino acid residue corresponding to the 42^{nd} position is Lys (K). In some embodiments, the Ig binding protein comprises one or more domains, wherein at least one domain comprises or essentially consists of or consists of an amino acid sequence of any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19 or an amino acid with at least 95%, at least 96%, at least 97%, at least 98%, or at least 99% identity to any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19 wherein the amino acid residue corresponding to the 5^{th} position is Phe (F) and the amino acid residue corresponding to the 8th position is lie (I) and the amino acid residue corresponding to the 28^{th} position is His (H), and the amino acid residue corresponding to the 42^{nd} position is Lys (K).

Affinity to Immunoglobulin. All Ig binding proteins or Ig binding domains as described herein bind to Immunoglobulin with a dissociation constant K_{D} preferably below 200 nM, or below 100 nM, even more preferably 10 nM or less. In some embodiments, the Ig binding protein or Ig binding domain binds to Ig, in particular IgG₁, IgG₂, IgG₄, IgM, and/or IgA, Ig fragments (thereof), Fc fragments, Fab fragments, fusion proteins comprising an Ig region or an Fc region of an Ig, and conjugates comprising an Ig region with a dissociation constant K_{D} preferably below 200 nM, or below 100 nM, even more preferably 10 nM or less. Methods for determining binding affinities or binding capacities of Ig binding proteins or domains, i.e. for determining the dissociation constant K_{D}, are known to a person of ordinary skill in the art and can be selected for instance from the following methods known in the art: Surface Plasmon Resonance (SPR) based technology, kinetic exclusion analysis (KinExA assay), Bio-layer interferometry (BLI), enzyme-linked immunosorbent assay (ELISA), flow cytometry, isothermal titration calorimetry (ITC), analytical ultracentrifugation, radioimmunoassay (RIA or IRMA) and enhanced chemiluminescence (ECL). Some of the methods are described further in the Examples. Typically, the dissociation constant K_{D} is determined at 20° C., 25° C., or 30° C., preferably at 20° C., 25° C., or 30° C. by SPR. If not specifically indicated otherwise, the K_{D} values recited herein are determined at 22° C.+/−3° C. by surface plasmon resonance spectroscopy. In one embodiment, the Ig binding protein has a dissociation constant K_{D} to human IgG₁ in the range between 0.1 nM and 100 nM, preferably between 0.1 nM and 50 nM. Preferably, the K_{D} in the range between 0.1 nM and 100 nM, preferably between 0.1 nM and 50 nM, means a K_{D} as determined by SPR, more preferably as determined by SPR at 20° C., 25° C., or 30° C.

High alkaline stability of Iq binding proteins. The Ig binding proteins and Ig binding domains of the invention provide surprisingly particularly good alkaline stability, shown as remaining IgG binding of at least about 80% after long term incubation at 1 M NaOH, as shown in the Examples, in addition to high dynamic binding capacities (DBC) of more than 60 mg/ml at 6 min residence time. The alkaline stability of the Ig binding protein or Ig binding domain is determined by comparing the loss in Ig binding activity. In some embodiments, the alkaline liquid comprises 0.1-1.0 M NaOH or KOH, preferably 0.5-1 M NaOH or KOH. Due to the high alkaline stability of the Ig binding proteins and Ig binding domains of the invention, an alkaline liquid with pH higher than 13 can be used for cleaning affinity matrices with immobilized Ig binding proteins or immobilized Ig binding domains of the invention. In some embodiments, the alkaline stability of the Ig binding protein or Ig binding domain is determined by comparing the loss in Ig binding activity after at least 37 h incubation in 1 M NaOH (see Examples). In some embodiments, the alkaline stability of the Ig binding protein or Ig binding domain is determined by comparing the loss in Ig binding activity after very long incubation in alkaline solution, e.g. for at least 2 days (at least 48 h) incubation in 1 M NaOH (see Examples), reflecting an extraordinary stability of the Ig binding proteins as described herein.

The Ig binding proteins and Ig binding domains of the invention are stable under alkaline conditions, in particular under alkaline conditions of 1 M NaOH for at least 37 h. In preferred embodiments, the Ig binding protein or Ig binding domain of the invention is stable under alkaline conditions, in particular under alkaline conditions of 1 M NaOH for at least 48 h, more preferably for at least 50 h.

The Ig binding proteins and Ig binding domains of the invention are alkaline-stable ligands for immunoglobulins. The Ig binding proteins and Ig binding domains of the invention retain binding capacity (or binding affinity) for immunoglobulin after exposure to 1 M NaOH for at least 37 h. As further described herein, the Ig binding proteins and Ig binding domains of the invention retain at least about 80% or at least about 83% or at least about 90% binding capacity for immunoglobulin after exposure to extreme alkaline conditions as described herein.

In further preferred embodiments, the Ig binding proteins and Ig binding domains of the invention retain at least about 80% binding capacity for immunoglobulin after exposure to extreme alkaline conditions (1 M NaOH for at least 37 h). In various embodiments, the Ig binding proteins and Ig binding domains of the invention retain binding capacity for immunoglobulin as described above when immobilized to a solid support, preferably to a solid support of an affinity separation matrix.

As further described herein, the Ig binding proteins and Ig binding domains of the invention are typically stable under alkaline conditions at room temperature. The term room temperature may include temperatures between 15° C. and 25° C., more specifically temperatures between 20° C. and 25° C. In various embodiments, the Ig binding protein or Ig binding domain of the invention is stable under alkaline conditions at 22° C.±3° C.

In various embodiments, the alkaline stability of the Ig binding protein or Ig binding domain as described above means alkaline stability of the Ig binding protein or Ig binding domain immobilized to a solid support, preferably to a solid support of an affinity separation matrix.

Hence, in various embodiments, the alkaline stability of the Ig binding protein or Ig binding domain is determined by comparing the loss in Ig binding activity or Ig binding capacity of the Ig binding protein or Ig binding domain when immobilized to a solid support, preferably to a solid support of an affinity separation matrix. Hence, in other embodiments, the alkaline stability of the Ig binding protein or Ig binding domain is determined by comparing the Ig binding activity of the Ig binding protein or Ig binding domain to a reference protein after alkaline treatment for a prolonged time when immobilized to a solid support.

The binding capacity or binding affinity for immunoglobulin of the Ig binding protein or Ig binding domain of the present invention can be evaluated by a skilled person using methods well known in the art, in particular methods for determining the dissociation constant K_{D} as described elsewhere herein. In various embodiments, the binding capacity or binding affinity for immunoglobulin of the Ig binding protein or Ig binding domain of the present invention is determined using Surface Plasmon Resonance (SPR) spectroscopy, as also described elsewhere herein. In other embodiments, the binding capacity or binding affinity for immunoglobulin of the Ig binding protein or Ig binding domain of the present invention is determined using kinetic exclusion analysis (KinExA assay), or enzyme-linked immunosorbent assay (ELISA), as described elsewhere herein.

The binding capacity or binding affinity for immunoglobulin of the Ig binding protein or Ig binding domain of the present invention can be assessed for each candidate ligand before and after exposure to alkaline conditions as described herein.

Multimers. In one embodiment, the Ig binding protein comprises 1, 2, 3, 4, 5, or 6 Ig binding domains linked to each other, i.e. the Ig binding protein can be, for example, a monomer, a dimer, a trimer, a tetramer, a pentamer, or a hexamer. A multimer may comprise two, three, four, five, or even more binding domains. Multimers of the invention are fusion proteins generated artificially, generally by recombinant DNA technology well-known to a skilled person.

In some embodiments, the multimer is a homo-multimer, e.g. the amino acid sequences of all Ig binding domains of the Ig binding protein are identical. In some embodiments, the multimer is a hetero-multimer, e.g. at least one Ig binding domain has a different amino acid sequence than the other Ig binding domains within the Ig-binding protein.

A multimer may comprise two or more Ig binding domains, wherein said Ig binding domains preferably comprise or essentially consist of an amino acid sequence as described above.

In some preferred embodiments, the multimer is a pentamer. The present invention provides pentamers comprising monomers of any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19. In various embodiments, an Ig binding protein of the invention is a pentamer comprising five Ig binding domains, wherein each of the five Ig binding domains corresponds to an Ig binding protein having at least 89.5% amino acid identity to any one of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19. wherein the amino acid residue corresponding to the 5^{th} position is Phe (F) and the amino acid residue corresponding to the 8th position is lie (I) and the amino acid residue corresponding to the 28^{th} position is His (H), and the amino acid residue corresponding to the 42^{nd} position is Lys (K), and wherein the pentameric Ig binding protein is stable under alkaline conditions.

In preferred embodiments, an Ig binding protein of the invention is a pentamer comprising five Ig binding domains, wherein each of the five Ig binding domains corresponds to an Ig binding protein having at least 89.5% amino acid identity to any one of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19. wherein the amino acid corresponding to position 5 is phenylalanine (F), the amino acid corresponding to position 8 is isoleucine (I), the amino acid corresponding to position 28 is histidine (H), and the amino acid corresponding to position 42 is lysine (K), and wherein the pentameric Ig binding protein is stable under alkaline conditions of 1 M NaOH for at least 37 h. In other preferred embodiments, an Ig binding protein of the invention is a pentamer comprising five Ig binding domains, wherein each of the 5 Ig binding domains corresponds to an Ig binding protein having at least 89.5% amino acid identity to any one of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19. wherein the amino acid corresponding to position 5 is phenylalanine (F), the amino acid corresponding to position 8 is isoleucine (I), the amino acid corresponding to position 28 is histidine (H), and the amino acid corresponding to position 42 is lysine (K), and wherein the pentameric Ig binding protein is stable under alkaline conditions of 1 M NaOH for at least 37 h, and wherein the pentameric Ig binding protein allows elution of the target at mild elution conditions of an pH of at least 4.0. In various embodiments, the pentameric Ig binding protein is stable under alkaline conditions of 1 M NaOH for at least 37 h, and allows elution of an Ig target (in particular an IgG) at elution conditions of at least about pH 4.3, preferably at least about pH 4.5. In further embodiments, the pentameric Ig binding protein is stable under alkaline conditions of 1 M NaOH for at least 37 h, and allows elution of the Ig target (in particular an IgG) at elution conditions of above pH 4.5.

In some specific embodiments, the Ig binding protein is a pentamer comprising the sequence of SEQ ID NO: 7, SEQ ID NO: 8, SEQ ID NO: 9, SEQ ID NO: 10, SEQ ID NO: 11, SEQ ID NO: 12, SEQ ID NO: 14, SEQ ID NO: 16, SEQ ID NO: 18, and SEQ ID NO: 20.

In other embodiment, the Ig binding protein is a pentamer comprising the sequence with at least 89.5% or at least 95% identity to any one of SEQ ID NO: 7, SEQ ID NO: 8, SEQ ID NO: 9, SEQ ID NO: 10, SEQ ID NO: 11, SEQ ID NO: 12, SEQ ID NO: 14, SEQ ID NO: 16, SEQ ID NO: 18, and SEQ ID NO: 20, provided that in each monomer of the multimer, the amino acid residue corresponding to the 5^{th} position is Phe (F) and the amino acid residue in each monomer of the multimer corresponding corresponding to the 8th position is lie (I) and the amino acid residue corresponding to the 28^{th} position is His (H), and the amino acid residue corresponding to the 42^{nd} position is Lys (K).

In preferred embodiments, the first monomer of the multimer is deleted at position 1 and position 2, thereby the positions of the amino acids corresponding to position 9, 11, 15 are as shown in Table 1.

TABLE 1
Positions in a pentamer as exemplified by SEQ ID
NOs: 7-12, 14, 16, and 18 (monomer 1:
56 amino acids, monomer 2, 3, 4, 5: 58 amino acids)

| | | Position 9, | Position |
|---|---|---|---|
| | Positions 5, 8, | 11, 15²⁾ are | 4, 7³⁾ are |
| | 28, 42¹⁾ are | corresponding | corresponding |
| | corresponding | to | to |
| Monomer | to position | position | position |
| 1^{st} Monomer | 3, 6, 26, 40 | 7, 9, 13 | 2,5 |
| 2^{nd} Monomer | 61, 64, 84, 98 | 65, 67, 71 | 60, 63 |
| 3^{rd} Monomer | 119, 122, 142, 156 | 123, 125, 129 | 118, 121 |
| 4^{th} Monomer | 177, 180, 200, 214 | 181, 183, 187 | 176, 179 |
| 5^{th} Monomer | 235, 238, 258, 272 | 239, 241, 245 | 234, 237 |

¹⁾These positions describe the amino acid residues at positions 5, 8, 28, and 42 described elsewhere herein in relation to SEQ ID NOs: 1-6, 13, 15, 17, and 19.
²⁾These positions describe the amino acid residues at positions 9, 11, and 15 described elsewhere herein in relation to, e.g., SEQ ID NOs: 1-6, 13, 15, 17, and 19.
³⁾These positions describe the amino acid residues at positions 4 and 7 described elsewhere herein in relation to, e.g., SEQ ID NOs: 1-6, 13, 15, 17 and 19.

The present invention encompasses multimeric forms of one or more monomers of any one of SEQ ID NOs: 1-6, 13, 15, 17, and 19 other than pentamers. Such other multimeric forms include in particular trimers, tetramers, and hexamers. In such multimeric forms, the position numbering depicted in the above Table 1 applies accordingly.

In some preferred embodiments, the multimer is a trimer comprising monomers of any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19. or Ig binding proteins with at least 89.5% identity thereto, respectively, as described elsewhere herein.

In some preferred embodiments, the multimer is a tetramer comprising monomers of any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19. or Ig binding proteins with at least 89.5% identity thereto, respectively, as described elsewhere herein.

In some preferred embodiments, the multimer is a hexamer comprising monomers of any of SEQ ID NO: 1, SEQ ID NO: 2, SEQ ID NO: 3, SEQ ID NO: 4, SEQ ID NO: 5, SEQ ID NO: 6, SEQ ID NO: 13, SEQ ID NO: 15, SEQ ID NO: 17, and SEQ ID NO: 19, or Ig binding proteins with at least 89.5% identity thereto, respectively, as described elsewhere herein.

Preferably, multimers of the present invention are based on one single type of monomer, the multimers are reflected in SEQ ID NOs: 7-12, 14, 16, and 18, or Ig binding proteins with at least 89.5% identity thereto, as described elsewhere herein.

In some embodiments, multimers of the present invention are based on more than one single type of monomer, such as the multimer reflected in SEQ ID NO: 20, or Ig binding proteins with at least 89.5% identity thereto, as described elsewhere herein. The present invention encompasses a pentamer based on SEQ ID NO: 19, wherein each of the five Ig binding domains corresponds to an Ig binding protein having at least 89.5% amino acid identity to SEQ ID NO: 19 as described elsewhere herein, except that in one of the two terminal domains, the amino acid corresponding to position 58 of SEQ ID NO: 19 is lysine (K), while the amino acid corresponding to position 58 of SEQ ID NO: 19 in all other domains is proline (P). As will be appreciated by one skilled in the art, all of the embodiments pertaining to the monomer of SEQ ID NO: 19 described elsewhere herein apply to the pentamer based on SEQ ID NO: 19 described here. In preferred embodiments, in the other terminal domain (i.e., the other of the said two terminal domains), the amino acid positions corresponding to positions 1 and 2 of SEQ ID NO: 19 are deleted (as reflected in SEQ ID NO: 20). More preferably, the said one of the two terminal domains carrying a lysine (K) at the position corresponding to position 58 of SEQ ID NO: 19 is the C-terminal domain of the pentamer (as reflected in SEQ ID NO: 20). The present invention encompasses a pentamer with at least 89.5% amino acid identity to SEQ ID NO: 20, wherein in one of the two terminal domains, the amino acid corresponding to position 58 of SEQ ID NO: 19 is lysine (K), while the amino acid corresponding to position 58 of SEQ ID NO: 19 in all other domains is proline (P). As will be appreciated by one skilled in the art, otherwise all of the embodiments pertaining to the pentamer of SEQ ID NO: 20 described elsewhere herein apply to the pentamer of SEQ ID NO: 20 described here. In particular, in the said pentamer of SEQ ID NO: 20, each of the five domains has a (F) at the position corresponding to position 5 of SEQ ID NO: 19, has an (I) at the position corresponding to position 8 of SEQ ID NO: 19, has a (H) at the position corresponding to position 28 of SEQ ID NO: 19, has a (k) at the position corresponding to position 42 of SEQ ID NO: 19. In preferred embodiments, in each of the five domains, the amino acid corresponding to position 5 is F, the amino acid corresponding to position 8 is I, the amino acid corresponding to position 28 is H, the amino acid corresponding to position 42 is K, the amino acid corresponding to position 4 is glutamine (Q), the amino acid corresponding to position 7 is lysine (K), the amino acid corresponding to positions 9 and 11 is alanine (A), and the amino acid corresponding to position 15 is glutamic acid (E). In particularly preferred embodiments, in the other terminal domain (i.e., the other of the said two terminal domains, which is not carrying a (K) at the position corresponding to position 58 of SEQ ID NO: 19), the amino acid positions corresponding to positions 1 and 2 of SEQ ID NO: 19 are deleted (which amino acid positions correspond to positions 1 and 2 of SEQ ID NO: 20). More preferably, the said one of the two terminal domains carrying a (K) at the terminal position (which is the position corresponding to position 58 of SEQ ID NO: 19) is the C-terminal domain of the pentamer (as reflected in SEQ ID NO: 20).

Further, in preferred embodiments of the invention, in multimeric forms, the first monomer has the first and second amino acid residue present in the corresponding monomer deleted, as reflected in SEQ ID NOs: 7-12, 14, 16, 18, 20, or Ig binding proteins with at least 89.5% identity thereto, respectively, as described elsewhere herein.

As described elsewhere herein, the present invention encompasses monomers of SEQ ID NOs: 1-6, 13, 15, 17, 19, or Ig binding proteins with at least 89.5% identity thereto, respectively, which incorporate the amino acid substitutions at positions 9, 11, and 15 described in relation to SEQ ID NO: 13, and/or which incorporate the amino acid substitutions at positions 4 and 7 described in relation to SEQ ID NO: 15.

The present invention further encompasses multimers according to any one of SEQ ID NOs: 7-12, 14, 16, 18, and 20, or Ig binding proteins with at least 89.5% identity thereto, respectively, which incorporate the amino acid substitutions at positions 9, 11, and 15 described in relation to SEQ ID NO: 13, and/or which incorporate the amino acid substitutions at positions 4 and 7 described in relation to SEQ ID NO: 15. With regard to the corresponding position numbering in such multimers of the invention, the identification of positions 4, 7, 9, 11, and 15 in the 1^{st}, 2^{nd}, 3^{rd}, 4^{th} and 5^{th} monomer as described in the above Table 1 applies.

Linker. In various embodiments, the one or more Ig binding domains are directly linked to each other. In other embodiments, the one or more Ig binding domains are linked to each other with one or more linkers. Preferred in these typical embodiments are peptide linkers. This means that the peptide linker is one or more amino acids, e.g. an amino acid sequence, that connects a first Ig binding domain with a second Ig binding domain. The peptide linker is connected to the first Ig binding domain and to the second Ig binding domain by a peptide bond between the C-terminal and N-terminal ends of the domains, thereby generating a single, linear polypeptide chain. In some embodiments, the multimer of Ig binding proteins comprises one or more linkers connecting the Ig binding domains wherein the linkers are identical or different.

Affinity separation matrix. In another embodiment the present invention is directed to an affinity separation matrix, comprising an Ig binding protein or Ig binding domain of the previous embodiments.

In preferred embodiments, the affinity separation matrix is a solid support. The affinity separation matrix comprises at least one Ig binding protein or Ig binding domain as described above.

An affinity matrix is useful for separation of immunoglobulins and should retain the Ig binding property even after highly alkaline conditions as applied during cleaning processes. Such cleaning of matrices is essential for long-term repeated use of matrices.

Solid support matrices for affinity chromatography are known in the art and include for example but are not limited to, agarose and stabilized derivatives of agarose (e.g. PraestoPure, Praesto Jetted A50, Praesto Jetted A50 HipH, Mabselect, PrismA, Sepharose 6B, CaptivA, rPROTEIN A Sepharose Fast Flow, MabCapturCand other), cellulose or derivatives of cellulose, controlled pore glass such as. ProSep vA Ultra, monolith such as convective interactive media (CIM) monolith, 3D printed monolith adsorption (PMA) column), silica, zirconium oxide (e.g. CM Zirconia or CPG), titanium oxide, or synthetic polymers (e.g. hydroxapaptite such as UNOsphere SUPrA polystyrene such as Poros 50A or Poros MabCapture A, polyvinylether, polyvinyl alcohol, monodisperse polyacrylate resin (e.g. UniMab, UniMabPro), polymethacrylate such as Toyopearl, polyhydroxyalkyl acrylates, polyhydroxyalkyl methacrylates, polyacrylamides, polymethacrylamides etc) and hydrogels of various compositions. In certain embodiments the support comprises a polyhydroxy polymer, such as a polysaccharide. Examples of polysaccharides suitable for supports include but are not limited to agar, agarose, dextran, starch, cellulose, pullulan, etc, and stabilized variants of these.

The formats for solid support matrices can be of any suitable well-known kind. Such solid support matrix for coupling the Ig binding protein or Ig binding domain as described herein might comprise for example, one of the following: columns, capillaries, particles, membranes, filters, monoliths, fibers, pads, gels, slides, plates, cassettes, or any other format commonly used in chromatography and known to someone skilled in the art.

In one embodiment, the matrix is comprised of substantially spherical particles, also known as beads, for example Sepharose or Agarose beads or monodisperse polyacrylate beads.

Suitable particle sizes may be in the diameter range of 5-500 μm, such as 10-100 μm, such as 20-80 μm, such as 40-70 μm. Matrices in particle form can be used as a packed bed or in a suspended form including expanded beds.

In an alternative embodiment, the solid support matrix is a membrane, for example a hydrogel membrane. In some embodiments, the affinity purification involves a membrane as matrix to which the Ig binding protein or Ig binding domain of the one embodiment is covalently bound.

The solid support can also be in the form of a membrane in a cartridge.

In some embodiments, the affinity purification involves a chromatography column containing a solid support matrix to which the Ig binding protein or Ig binding domain of the one embodiment is covalently bound.

Immobilization to a solid support. In embodiments of the invention, the Ig binding protein or Ig binding domain is conjugated to a solid support. In some embodiments of the invention, the Ig binding protein or Ig binding domain may comprise additional amino acid residues at the N- and/or C-terminal end. The Ig binding protein or Ig binding domain of the invention may be attached to a suitable solid support matrix via conventional coupling techniques. Methods for immobilization of protein ligands to solid supports are well-known in this field and easily performed by the skilled person in this field using standard techniques and equipment. In some embodiments, the coupling may be a multipoint coupling, for example via several lysines, or a single point coupling, for example via cysteine.

In some embodiments, the alkaline stable Ig binding protein or Ig binding domain comprises an attachment site for covalent attachment to a solid phase (matrix). Site-specific attachment sites comprise natural amino acids, such as cysteine or lysine, which enable specific chemical reactions with a reactive group of the solid phase or a linker between the solid phase and the protein.

In some embodiments, the attachment site may be directly at the C- or N-terminal end of the Ig binding protein or Ig binding domain. In some embodiments, a single cysteine is located at the C-terminal end for site-specific immobilization of the Ig binding protein or Ig binding domain.

An advantage of having a C-terminal cysteine is that coupling of the Ig binding protein or Ig binding domain can be achieved through reaction of the cysteine thiol with an electrophilic group on a support resulting in a thioether bridge coupling. This provides excellent mobility of the coupled protein which provides increased binding capacity.

In other embodiments, the attachment site may be located in the Ig binding protein or Ig binding domain, for example, at a position corresponding to position 43 or position 46 of, e.g., any of SEQ ID NOs: 1-6, 13, 15, 17, 19.

In other embodiments, there may be a linker between the N- or C-terminus and the attachment site. In some embodiments of the invention, the Ig binding protein or Ig binding domain may comprise a N- or C-terminal amino acid sequence of 3-20 amino acids, preferably of 4-10 amino acids, with a terminal cysteine. Amino acids for a terminal attachment site may be selected from the group of proline, glycine, alanine, and serine, with a single cysteine at the C-terminal end for coupling.

In some embodiments of the invention, the Ig binding protein or Ig binding domain may also comprise additional amino acid residues at the N- and/or C-terminal end, such as for example a leader sequence at the N-terminal end and/or a coupling sequence with or without a tag at the N- or C-terminal end.

Use of the Iq binding protein. In a one embodiment the present invention is directed to the use of the Ig binding protein or Ig binding domain of the one embodiment or an affinity matrix of the one embodiment for affinity purification of immunoglobulins or variants thereof, i.e. the Ig binding protein or Ig binding domain of the invention is used for affinity chromatography. In some embodiments, the Ig binding protein or Ig binding domain of the invention is immobilized onto a solid support as described in the one embodiment of the invention.

Method of affinity purification of immunoglobulins. In one embodiment the present invention is directed to a method of affinity purification of immunoglobulins, the method comprising the following steps:
(a) providing a liquid (sample) that contains an Ig such as IgG₁, IgG₂, IgG₄, IgM, IgA, Ig fragments, Fc fragments, or Fab fragments (including fusion proteins and conjugates, as defined above);
(b) providing an affinity separation matrix comprising an immobilized Ig binding protein or Ig binding domain as described above immobilized to said affinity separation matrix;
(c) contacting said liquid with said affinity separation matrix, under conditions that permit binding of the at least one Ig binding protein or Ig binding domain as described above to an Ig; and
(d) eluting said Ig from said matrix, thereby obtaining an eluate containing said Ig.

In some embodiments, the method of affinity purification may further comprise one or more washing steps carried out between steps (c) and (d) under conditions sufficient to remove from the affinity separation matrix some or all molecules that are non-specifically bound thereto.

Non-specifically bound means any binding that does not involve an interaction between the at least one Ig binding protein or Ig binding domain and an g.

Affinity separation matrices suitable for the disclosed uses and methods are those matrices according to the embodiments described above and as known to someone skilled in the art.

In some embodiments, the elution of the immunoglobulin from (the matrix comprising) the Ig binding protein or Ig binding domain in step (d) is effected through a change in pH and/or a change in salt concentration. In general, suitable conditions for performing the method of affinity purification are well known to someone skilled in the art. In some embodiments, the disclosed uses or methods of affinity purification comprising the disclosed Ig binding proteins or Ig binding domains may provide elution of at least about 95%, at least about 96%, at least about 97%, at least about 98%, at least about 99%, or 100% of Ig containing proteins at a pH of equal or greater than pH 4.0. Due to the high stability of the Ig binding proteins and Ig binding domains of the invention, solutions with greater than or equal to pH 4.0 can be used for the elution of Ig proteins (see Examples).

In some embodiments, in step (d) of the method of affinity purification more than about 95% of the protein comprising the Ig sequence (e.g. antibody) is eluted at pH 4.0 (or higher) from the matrix comprising the immobilized Ig binding protein or Ig binding domain as described above. In some embodiments, a further step (e) for efficient cleaning of the affinity matrix is added, preferably by using an alkaline liquid, for example, with pH of 13-14. In certain embodiments, the cleaning liquid comprises 0.1-1.0 M NaOH or KOH, preferably 0.5-1 M NaOH or KOH. Due to the high alkaline stability of the Ig binding proteins or Ig binding domains of the invention, such strong alkaline solution can be used for cleaning purposes. After cleaning the affinity purification matrix with an alkaline cleaning liquid, in some embodiments, at least about 80% of the Ig binding protein or Ig binding domain have Ig binding activity if incubated for at least 37 h at 1 M NaOH. In some embodiments, the Ig-binding capacity of the Ig binding protein or Ig binding domain is at least about 80% or at least about 90% of the Ig binding capacity before the incubation under alkaline conditions, for example, as determined by the remaining Ig-binding capacity after at least 37 h incubation in 1 M NaOH.

The present invention further provides a method of isolating an immunoglobulin, comprising the steps (a) contacting a liquid sample comprising an immunoglobulin with a separation matrix comprising a plurality of Ig binding proteins or Ig binding domains (coupled to a solid support); (b) washing the separation matrix with a washing liquid such as 1 M NaOH; (c) eluting the immunoglobulin from the separation matrix with pH 4.0 or pH 4.5 or higher; and (d) obtaining the immunoglobulin. In various preferred embodiments, the elution is performed under acidic conditions of about pH 4.0, pH 4.3, or about pH 4.5. In various embodiments, the elution may be performed at a pH above pH 4.5.

Nucleic acid molecule. In one embodiment, the present invention is directed to a nucleic acid molecule, preferably an isolated nucleic acid molecule, encoding an Ig binding protein or Ig binding domain as disclosed above. In one embodiment, the present invention is directed to a vector comprising the nucleic acid molecule. A vector means any molecule or entity (e.g., nucleic acid, plasmid, bacteriophage or virus) that can be used to transfer protein coding information into a host cell. In one embodiment, the vector is an expression vector.

In one embodiment, the present invention is directed to an expression system which comprises a nucleic acid or a vector as disclosed above, for example a prokaryotic host cell, for example E. coli, or a eukaryotic host, for example yeast Saccharomyces cerevisiae or Pichia pastoris or mammalian cells such as CHO cells.

Method for the production of a Iq binding protein. In one embodiment the present invention is directed to a method for the production of a Ig binding protein or Ig binding domain of the invention, comprising the step(s): (a) culturing the host cell of the one embodiment under suitable conditions for the expression of the binding protein or Ig binding domain in order to obtain said Ig binding protein or Ig binding domain; and (b) optionally isolating said Ig binding protein or Ig binding domain. Suitable conditions for culturing a prokaryotic or eukaryotic host are well-known to the person skilled in the art.

Ig binding molecules of the invention may be prepared by any of the many conventional and well-known techniques such as plain organic synthetic strategies, solid phase-assisted synthesis techniques or by commercially available automated synthesizers. On the other hand, they may also be prepared by conventional recombinant techniques alone or in combination with conventional synthetic techniques.

One embodiment of the present invention is directed to a method for the preparation of a Ig binding protein or Ig binding domain according to the invention as detailed above, said method comprising the following steps: (a) preparing a nucleic acid encoding an Ig binding protein or Ig binding domain as defined above; (b) introducing said nucleic acid into an expression vector; (c) introducing said expression vector into a host cell; (d) cultivating the host cell; (e) subjecting the host cell to culturing conditions under which an Ig binding protein or Ig binding domain is expressed, thereby (e) producing an Ig binding protein or Ig binding domain as described above; optionally (f) isolating the Ig binding protein or Ig binding domain produced in step (e); and (g) optionally conjugating the Ig binding protein or Ig binding domain to solid matrices as described above. In a further embodiment of the present invention the production of the Ig binding protein or Ig binding domain is performed by cell-free in vitro transcription/translation.

### EXAMPLES

The following Examples are provided for further illustration of the invention. The invention, however, is not limited thereto, and the following Examples merely show the practicability of the invention on the basis of the above description.

### Example 1. Expression

Ligands (for example, 224785 (SEQ ID NO: 12), 224770 (SEQ ID NO: 9), 224771 (SEQ ID NO: 10), 224772 (SEQ ID NO: 8), 224777 (SEQ ID NO: 7), 228302 (SEQ ID NO: 18), 230620 (SEQ ID NO: 20)) were expressed in Escherichia coli BL21(DE3) using a pNP-016 vector system under regulation of a T7 promoter. Proteins were produced in soluble form after induction by lactose included in the medium (autoinduction medium). BL21 (DE3) competent cells were transformed with the expression plasmid, spread onto selective agar plates (kanamycin) and incubated over night at 37° C. Precultures were inoculated from single colony in 50 ml 2×YT medium supplemented with 50 μg/ml kanamycin and cultured for 7 h at 37° C. in shake flasks. For main cultures 350 mL autoinduction medium (modified H15 medium consisting of 2% glucose, 5% yeast extract, 0.89% glycerol, 0.76% lactose, 250 mM MOPS, 202 mM TRIS, 10 mM MgSO4, pH 7.4, antifoam SE15) supplemented with 50 μg/ml kanamycin and trace elements were inoculated to an OD600 of 0.3 and incubated in 2.5 L Ultra Yield™ flasks at 37° C. in an orbital shaker. Recombinant protein expression was induced by metabolizing glucose and subsequently allowing lactose to enter the cells. Cells were grown over night for approximately 18 hours to reach a final OD600 of about 40-50. Before the harvest, the OD600 was measured, samples adjusted to 0.6/OD600 were withdrawn, pelleted and frozen at −20° C. To collect biomass cells were centrifuged at 12,000×g for 20 min at 22° C. Pellets were weighed (wet weight) and stored at −20° C. before processing.

### Example 2: SDS-PAGE Analysis of Expression and Acidic Solubility

Samples were resuspended in 90 pl extraction buffer (PBS supplemented with 0.2 mg/ml Lysozyme, 0.5× BugBuster, 6 mM MgSO₄, 6 mM MgCl₂, 15 U/mL Benzonase) and solubilized by agitation in a thermomixer at 850 rpm, rt for 15 min with a subsequent incubation at −80° C. for 15 min. After thawing, soluble proteins were separated from insoluble proteins by centrifugation (16,000×g, 2 min, rt). Supernatant was withdrawn (soluble fraction) and the pellet (insoluble fraction) was resuspended in equivalent amount of urea buffer (8 M urea, 0.2 M Tris, 20 mM EDTA, pH 7.0). 35 pl were taken both from the soluble and insoluble fraction, and 10 pl 5× sample buffer as well as 5 pl 0.5 M DTT were added. Samples were boiled at 95° C. for 5 min. Finally, 5 μl of those samples were applied to NuPage Novex 4-12% Bis-Tris SDS gels which were run in accordance to the manufacturer's recommendations and stained with Coomassie. Results: Mid- to high level expression was found under optimized conditions within the chosen period of time. All expressed Ig binding proteins showed acidic solubility.

### Example 3: Purification

Ig binding proteins were expressed in the soluble fraction of E. coli. The cells were resuspended in cell disruption buffer and lysed by an ultrasonic cell disruption system (Sonopuls HD 2200, Bandelin). Purification step was performed with IEC Sepharose SP-HP (Cytiva) using an AKTAvant system (Cytiva) according to the manufacturer's instructions using citric acid buffer at pH 3.0 (20 mM Citric acid, 1 mM EDTA, pH 3.0). Pure protein fractions were eluted by increasing sodium chloride concentration to 1 M with a linear gradient in 10 column volumes. Furth purification was performed by size exclusion chromatography (Superdex 75) according to manufactures instructions using citric acid buffer pH 6.0 (20 mM Citric acid, 150 mM NaCl, 1 mM EDTA, pH 6.0). Results: The purity of variants was 100% after SE-HPLC and >96% after RP HPLC.

### Example 4. The Ig Binding Proteins Bind to IgG with High Affinities

A sensor chip (Bruker) was equilibrated with surface plasmon resonance (SPR) running buffer. Surface-exposed carboxylic groups were activated by passing a mixture of EDC and NHS to yield reactive ester groups. 700-1500 RU on-ligand were immobilized on a flow cell, off-ligand was immobilized on another flow cell. Injection of ethanolamine after ligand immobilization removes non-covalently bound Ig binding protein. Upon ligand binding, protein analyte was accumulated on the surface increasing the refractive index. This change in the refractive index was measured in real time and plotted as response or resonance units (RU) versus time. The analytes were applied to the chip in serial dilutions with a suitable flow rate (pl/min). After each run, the chip surface was regenerated with regeneration buffer and equilibrated with running buffer. The control samples were applied to the matrix. Regeneration and re-equilibration were performed as previously mentioned. Binding studies were carried out by the use of the Bruker SPR-32 at 25° C.; data evaluation was operated via the Bruker evaluation software, provided by the manufacturer, by the use of the Langmuir 1:1 model (RI=0). Evaluated dissociation constants (K_{D}) were standardized against off-target and K_{D} values of Ig binding proteins for Cetuximab (IgG₁), Natalizumab (IgG₄), and Panitumab (IgG₂) in Table 2. 228302 (SEQ ID NO: 18) binds to IgG1 of Bevacizumab with K_{D} of 3.7 nM. 228302 binds to IgG1 of Trastuzumab with K_{D} of 7.6 nM.

TABLE 2
K_{D} values of Ig binding proteins for IgG

| | | comprises | vs. | vs. | vs. |
|---|---|---|---|---|---|
| | SEQ ID | SEQ ID | hIgG₁ | hIgG₂ | hIgG₄ |
| Ligand | NO: | NO: | (nM) | (nM) | (nM) |
| 224777 | 7 | 1 | 2.4 | 5.8 | 2.0 |
| 224772 | 8 | 2 | <0.5 | <0.5 | <0.5 |
| 224770 | 9 | 3 | 2.8 | 9.1 | 3.0 |
| 224771 | 10 | 4 | <0.5 | 2.0 | <0.5 |
| 224785 | 12 | 6 | <0.5 | 1.7 | <0.5 |
| 228302 | 18 | 17 | 1.7 | 24 | 3.3 |

COUPLING: Purified ligands (224770, 224771, 224772, 224777, 224785, 228302, 230620) were coupled to agarose-based chromatography beads (Praesto Jetted Expoxy 50, Purolite) according to the manufacturer's instructions (20 mg/ml matrix). The coupling efficiency was at least 90% for all Ig binding proteins.

DBC10%: Coupled resin was packed into super compact 5/50 column (Götec GmbH). Octagam was used as IgG sample (conc. 2.2 mg/ml; in 1×PBS, pH 7.3). Octagam sample was applied to the matrix comprising immobilized ligand until 10% target breakthrough at 6 min (or 5 min for 228302 in Table 3A) residence time. Unbound sample was washed with 1×PBS with 1 M NaCl, pH 7.3. Loaded antibody was quantified and calculated as dynamic binding capacity DBC10%. Results: The binding capacities (DBC10) for all ligands were about 60 mg/ml, as shown in Table 3A. Caustic stability reflects the remaining binding capacity (in %) compared to the binding capacity at 0 h.

Caustic stability. Columns were incubated with 1 M NaOH for 37.5 h and 50 h (Table 3A), for 50 h (Table 3B) at room temperature (22° C.+/−3° C.); 2 sets were measured. The Ig binding activity of the immobilized ligand was analyzed after incubation with 1 M NaOH. Results: After 37.5 h in 1 M NaOH, all variants showed a remaining IgG binding capacity (DBC10 in %) of at least about 80%. 224772 had a remaining IgG binding capacity (DBC10 in %) of 90%. Even after 50 h in extremely strong alkaline solution, 224770, 224772, and 224777 showed still about 75% of the initial remaining binding capacity for Ig (see Table 3A). 230620 had a remaining IgG (Trastuzumab) binding capacity (DBC10 in %) of about 91% (after 37.5 h, 1 M NaOH).

Multimers (trimer, tetramer, pentamer, hexamer) of SEQ ID NO: 6 show comparable binding activity after 50 h incubation at 1 M NaOH (see Table 3B). Similar observations are made with multimers (trimer, pentamer) of SEQ ID NOs: 2, 3, and 17 (data not shown).

Step pH elution: Step pH elution at pH 3.5, pH 3.7, and pH 4.0 with 0.1 M acetic acid for 15 CV at 1 CV/min followed by 10 CV 0.1 M phosphoric acid pH 1.7 (CIP) to elute hIgG (Load: 2.2 mg/mL Octagam, 6 min residence time) that was bound to the immobilized ligand. Results: For all variants tested, more than about 95% of the antibody was eluted at pH 4.0. For 224770 and 224777, 100% of the antibody was eluted at pH 4.0 (see Table 3A). For 228302, step pH elution at pH 3.7, pH 4.0, and pH 4.5 with 0.1 M acetic acid for 15 CV at 1 CV/min followed by 10 CV 0.1 M phosphoric acid pH 1.7 (CIP) to elute hIgG (Load: 2.2 mg/mL Octagam, 5 min residence time) that was bound to the immobilized ligand. About 92% of the antibody was eluted at pH 4.0. At pH 4.5, 228302 allowed an elution of 84% of the IgG.

Table 3. Affinity chromatography properties of ligands

TABLE 3A
DBC10, remaining IgG binding capacity, elution

| | | | DBC10% | Remaining IgG | | | |
|---|---|---|---|---|---|---|---|
| | | | 6 min | binding capacity | | | |
| | | | residence | after 37.5 h, | after 50 h, | Amount | |
| | | comprises | time | 1 M NaOH | 1 M NaOH | eluted IgG | |
| Ligand | SEQ ID NO: | SEQ ID NO: | (mg/ml) | (%) | (%) | pH 4.0 (%) | pH 3.7 (%) |
| 224770 | 9 | 3 | 60 | 83 | 74 | 100 | 0 |
| 224771 | 10 | 4 | 63 | 79 | 66 | 97 | 3 |
| 224772 | 8 | 2 | 65 | 90 | 80 | 97 | 3 |
| 224777 | 7 | 1 | 61 | 80 | 76 | 100 | 0 |
| 224785 | 12 | 6 | 61 | 78 | 63 | 94 | 6 |
| 228302 | 18 | 17 | 53* | n.d. | 75 | 92 | 8 |

*DBC10% was determined after 5 min residence time (mg/ml)

TABLE 3B
Remaining binding activity of multimers of SEQ ID
NO: 6 after incubation for 50 h at 1M NaOH

| | | | DBC10 | DBC10 after |
|---|---|---|---|---|
| | Multimer | | before | 50 h 1M |
| | of | | NaOH | NaOH 6 |
| | SEQ | Coupling | treatment (5 | min |
| | ID NO: | density | min residence | residence |
| CID | 6 | (mg/ml) | time, mg/ml) | time (mg/ml) |
| 229297 | trimer | 19 | 63 | 38 |
| 228296 | tetramer | 19 | 59 | 40 |
| 224785 | Pentamer | 19 | 56 | 41 |
| 228294 | hexamer | 20 | 61 | 41 |

SEQUENCES

SEQ ID NO: 1 (224777 monomer)
IAAQFDKIAQIAFYAILHLPNLTEEQRHAFIQSLRDDPSVSKEILAEAKKLNDAQAPK
SEQ ID NO: 2 (224772 monomer)
IAAQFDKIQQAAFYAILHLPNLTEEQRHAFIQSLRDDPSVSKEILAEAKKLNDAQAPK
SEQ ID NO: 3 (224770 monomer)
IAAQFDKIAQAAFYEILHLPNLTEEQRHAFIQSLRDDPSVSKEILAEAKKLNDAQAPK
SEQ ID NO: 4 (224771 monomer)
IAAQFDKIQQIAFYEILHLPNLTEEQRHAFIQSLRDDPSVSKEILAEAKKLNDAQAPK
SEQ ID NO: 5 (SEQ ID NO: 11 monomer)
IAAQFDKIAQAAFYAILHLPNLTEEQRHAFIQSLRDDPSVSKEILAEAKKLNDAQAPK
SEQ ID NO: 6 (224785 monomer)
IAAQFDKIQQAAFYEILHLPNLTEEQRHAFIQSLRDDPSVSKEILAEAKKLNDAQAPK
SEQ ID NO: 7 (224777 pentamer)
AQFDKIAQIAFYAILHLPNLTEEQRHAFIQSLRDDPSVSKEILAEAKKLNDAQAPKIAAQFDKIAQIAFYAILHL
PNLTEEQRHAFIQSLRDDPSVSKEILAEAKKLNDAQAPKIAAQFDKIAQIAFYAILHLPNLTEEQRHAFIQSLRD
DPSVSKEILAEAKKLNDAQAPKIAAQFDKIAQIAFYAILHLPNLTEEQRHAFIQSLRDDPSVSKEILAEAKKIND
AQAPKIAAQFDKIAQIAFYAILHLPNLTEEQRHAFIQSLRDDPSVSKEILAEAKKLNDAQAPK
SEQ ID NO: 8 (224772 pentamer)
AQFDKIQQAAFYAILHLPNLTEEQRHAFIQSLRDDPSVSKEILAEAKKLNDAQAPKIAAQFDKIQQAAFYAILHL
PNLTEEQRHAFIQSLRDDPSVSKEILAEAKKLNDAQAPKIAAQFDKIQQAAFYAILHLPNLTEEQRHAFIQSLRD
DPSVSKEILAEAKKLNDAQAPKIAAQFDKIQQAAFYALHLPNLTEEQRHAFIQSLRDDPSVSKEILAEAKKIND
AQAPKIAAQFDKIQQAAFYAILHLPNLTEEQRHAFIQSLRDDPSVSKEILAEAKKLNDAQAPK
SEQ ID NO: 9 (224770 pentamer)
AQFDKIAQAAFYEILHLPNLTEEQRHAFIQSLRDDPSVSKEILAEAKKLNDAQAPKIAAQFDKIAQAAFYEILHL
PNLTEEQRHAFIQSLRDDPSVSKEILAEAKKLNDAQAPKIAAQFDKIAQAAFYEILHLPNLTEEQRHAFIQSLRD
DPSVSKEILAEAKKLNDAQAPKIAAQFDKIAQAAFYEILHLPNLTEEQRHAFIQSLRDDPSVSKEILAEAKKLND
AQAPKIAAQFDKIAQAAFYEILHLPNLTEEQRHAFIQSLRDDPSVSKEILAEAKKLNDAQAPK
SEQ ID NO: 10 (224771 pentamer)
AQFDKIQQIAFYEILHLPNLTEEQRHAFIQSLRDDPSVSKEILAEAKKLNDAQAPKIAAQFDKIQQIAFYEILHL
PNLTEEQRHAFIQSLRDDPSVSKEILAEAKKLNDAQAPKIAAQFDKIQQIAFYEILHLPNLTEEQRHAFIQSLRD
DPSVSKEILAEAKKLNDAQAPKIAAQFDKIQQIAFYEILHLPNLTEEQRHAFIQSLRDDPSVSKEILAEAKKIND
AQAPKIAAQFDKI QQIAFYEILHLPNLTEEQRHAFIQSLRDDPSVSKEILAEAKKLNDAQAPK
SEQ ID NO: 11 (pentamer)
AQFDKIAQAAFYAILHLPNLTEEQRHAFIQSLRDDPSVSKEILAEAKKLNDAQAPKIAAQFDKIAQAAFYAILHL
PNLTEEQRHAFIQSLRDDPSVSKEILAEAKKLNDAQAPKIAAQFDKIAQAAFYAILHLPNLTEEQRHAFIQSLRD
DPSVSKEILAEAKKLNDAQAPKIAAQFDKIAQAAFYAILHLPNLTEEQRHAFIQSLRDDPSVSKEILAEAKKIND
AQAPKIAAQFDKIAQAAFYAILHLPNLTEEQRHAFIQSLRDDPSVSKEILAEAKKLNDAQAPK
SEQ ID NO: 12 (224785 pentamer)
AQFDKIQQAAFYEILHLPNLTEEQRHAFIQSLRDDPSVSKEILAEAKKLNDAQAPKIAAQFDKIQQAAFYEILHL
PNLTEEQRHAFIQSLRDDPSVSKEILAEAKKLNDAQAPKIAAQFDKIQQAAFYEILHLPNLTEEQRHAFIQSLRD
DPSVSKEILAEAKKLNDAQAPKIAAQFDKIQQAAFYEILHLPNLTEEQRHAFIQSLRDDPSVSKEILAEAKKLND
AQAPKIAAQFDKIQQAAFYEILHLPNLTEEQRHAFIQSLRDDPSVSKEILAEAKKLNDAQAPK
SEQ ID NO: 13 (monomer)
IAAQFDKIXQXAFYXILHLPNLTEEQRHAFIQSLRDDPSVSKEILAEAKKLNDAQAPK
X at position 9 may be A or Q, X at position 11 may be A or I, X at
position 15 may be A or E.
SEQ ID NO: 14 (pentamer)
AQFDKIXQXAFYXILHLPNLTEEQRHAFIQSLRDDPSVSKEILAEAKKLNDAQAPKIAAQFDKIXQXAFYXILHL
PNLTEEQRHAFIQSLRDDPSVSKEILAEAKKLNDAQAPKIAAQFDKIXQXAFYXILHLPNLTEEQRHAFIQSLRD
DPSVSKEILAEAKKLNDAQAPKIAAQFDKIXQXAFYXILHLPNLTEEQRHAFIQSLRDDPSVSKEILAEAKKIND
AQAPKIAAQFDKIXQXAFYXILHLPNLTEEQRHAFIQSLRDDPSVSKEILAEAKKLNDAQAPK
X at position 7, 65, 123, 181, and 239 may be A or Q, X at position 9, 67,
125, 183, and 241 may be A or I, X at position 13, 71, 129, 187, and 245
may be A or E.
SEQ ID NO: 15 (monomer)
IAAXFDXIXQXAFYXILHLPNLTEEQRHAFIQSLRDDPSVSKEILAEAKKLNDAQAPK
X at position 4 may be Q or K, X at position 7 may be K or E, X at position
9 may be A or Q, X at position 11 may be A or I, X at position 15 may be A
or E.
SEQ ID NO: 16 (pentamer)
AXFDXIXQXAFYXILHLPNLTEEQRHAFIQSLRDDPSVSKEILAEAKKLNDAQAPKIAAXEDXIXQXAFYXILHL
PNLTEEQRHAFIQSLRDDPSVSKEILAEAKKLNDAQAPKIAAXEDXIXQXAFYXILHLPNLTEEQRHAFIQSLRD
DPSVSKEILAEAKKLNDAQAPKIAAXFDXIXQXAFYXILHLPNLTEEQRHAFIQSLRDDPSVSKEILAEAKKLND
AQAPKIAAXFDXIXQXAFYXILHLPNLTEEQRHAFIQSLRDDPSVSKEILAEAKKLNDAQAPK
X at position 2, 60, 118, 176, and 234 may be Q or K, X at position 5, 63,
121, 179, and 237 may be K or E, X at position 7, 65, 123, 181, and 239 may
be A or Q, X at position 9, 67, 125, 183, and 241 may be A or I, X at
position 13, 71, 129, 187, and 245 may be A or E.
SEQ ID NO: 17 (228302 monomer)
IAAKFDEIAQAAFYEILHLPNLTEEQRHAFIQSLRDDPSVSKEILAEAKKLNDAQAPK
SEQ ID NO: 18 (228302 pentamer)
AKFDEIAQAAFYEILHLPNLTEEQRHAFIQSLRDDPSVSKEILAEAKKLNDAQAPKIAAKFDEIAQAAFYEILHL
PNLTEEQRHAFIQSLRDDPSVSKEILAEAKKLNDAQAPKIAAKFDEIAQAAFYEILHLPNLTEEQRHAFIQSLRD
DPSVSKEILAEAKKLNDAQAPKIAAKFDEIAQAAFYEILHLPNLTEEQRHAFIQSLRDDPSVSKEILAEAKKLND
AQAPKIAAKFDEIAQAAFYEILHLPNLTEEQRHAFIQSLRDDPSVSKEILAEAKKLNDAQAPK
SEQ ID NO: 19 (230620 monomer)
IAAQFDKIAQAAFYEILHLPNLTEEQRHAFIQSLRDDPSVSKEILAEAKKLNDAQAPP
SEQ ID NO: 20 (230620 pentamer)
AQFDKIAQAAFYEILHLPNLTEEQRHAFIQSLRDDPSVSKEILAEAKKLNDAQAPPIAAQFDKIAQAAFYEILHL
PNLTEEQRHAFIQSLRDDPSVSKEILAEAKKLNDAQAPPIAAQFDKIAQAAFYEILHLPNLTEEQRHAFIQSLRD
DPSVSKEILAEAKKLNDAQAPPIAAQFDKIAQAAFYEILHLPNLTEEQRHAFIQSLRDDPSVSKEILAEAKKIND
AQAPPIAAQFDKIAQAAFYEILHLPNLTEEQRHAFIQSLRDDPSVSKEILAEAKKLNDAQAPK

1. An Immunoglobulin (Ig) binding protein comprising an amino acid sequence with at least 89.5% amino acid identity to SEQ ID NO: 13, wherein the amino acid corresponding to position 5 is phenylalanine (F), the amino acid corresponding to position 8 is isoleucine (1), the amino acid corresponding to position 28 is histidine (H), and the amino acid corresponding to position 42 is lysine (K).

2. The Ig binding protein of claim 1, wherein the amino acid corresponding to position 9 is alanine (A) or glutamine (Q).

3. The Ig binding protein of claim 1, wherein the amino acid corresponding to position II is alanine (A) or isoleucine (I).

4. The Ig binding protein of claim 1, wherein the amino acid corresponding to position 15 is alanine (A) or glutamic acid (E).

5. The Ig binding protein of claim 1, wherein the Ig binding protein is multimeric and comprises at least 3 Ig binding proteins.

6. The Ig binding protein of claim 5, wherein the multimeric Ig binding protein is a pentamer.

7. The Ig binding protein of claim 1, wherein the Ig binding protein comprises any amino acid sequence selected from the group consisting of SEQ ID NOs: 1-14.

8. The Ig binding protein of claim 1, wherein said protein binds to one or more of IgG1, IgG2, IgG4, IgM, IgA, Ig fragments, Fe fragments, Fab fragments, fusion proteins comprising an Ig region, and conjugates comprising an Ig region.

9. The Ig binding protein of claim 1, wherein the Ig binding protein is immobilized to a solid support.

10. The Ig binding protein of claim 1, wherein the Ig binding protein is stable under alkaline conditions.

11. An affinity separation matrix comprising at least one Ig binding protein of claim 1.

12. (canceled)

13. A method for purifying a protein comprising an Fc region of an immunoglobulin (Ig), the method comprising:
(a) providing a sample, optionally a liquid sample, that contains a protein comprising an Fc region of an Ig;
(b) providing the affinity separation matrix according to claim 11;
(c) contacting said affinity separation matrix with the sample under conditions that permit binding of the at least one Ig binding protein of the affinity separation matrix to a protein comprising an Fc region of an Ig; and
(d) collecting, optionally eluting, said protein comprising an Fc region of an Ig from said affinity purification matrix,
thereby obtaining said protein comprising an Fc region of an Ig.

14. The method of claim 13, wherein in step (d) more than about 95% of the protein comprising the Ig sequence is eluted at pH 4.0 from the affinity separation matrix.

15. The Ig binding protein of claim 8, wherein the Ig binding protein binds to a protein comprising an 1c region or to an 1c fragment.

16. The Ig binding protein of claim 10, wherein the Ig binding protein is stable for at least 37 hours in I M NaOH.

17. The affinity separation matrix of claim 11, wherein the at least one Ig binding protein is coupled to the affinity separation matrix.

18. The method of claim 13, further comprising obtaining an eluate containing said protein comprising the Fc region of an Ig.
